(19) United States
(12) Patent Application Publication
(10) Pub. No.: US 2025/0340517 A1
(43) Pub. Date: Nov. 6, 2025

(54) HIGH-DIELECTRIC CONSTANT ZWITTERIONIC LIQUIDS

(71) Applicant: THE PENN STATE RESEARCH FOUNDATION, University Park, PA (US)

(72) Inventors: Ralph H. COLBY, University Park, PA (US); Robert J. HICKEY, University Park, PA (US); Wenwen MEI, University Park, PA (US)

(21) Appl. No.: 18/866,226

(22) PCT Filed: May 17, 2023
(86) PCT No.: PCT/US2023/022487

§ 371 Date: Nov. 15, 2024

Related U.S. Application Data
(60) Provisional application No. 63/342,723, filed on May 17, 2022.

(51) Int. Cl. C07D 233/61; C07C 303/22; C07C 309/14; C07D 233/60; C07D 295/108; C07D 295/15

(52) U.S. Cl. CPC C07D 233/61; C07C 303/22; C07C 309/14; C07D 233/60; C07D 295/108; C07D 295/15

(57) ABSTRACT

Disclosed herein are high dielectric constant zwitterionic compounds of Formula (I) and methods of making the same. These materials are liquid near room temperature, with unprecedentedly high dielectric constants, making them both novel and useful. Dielectric constant can be tuned by choice of anion, choice of cation, choice of cation tail, and length of the linker that covalently connects the anion to the cation.

### CROSS-REFERENCE TO RELATED APPLICATIONS

This application claims the benefit of priority to U.S. Provisional Application No. 63/342,723, filed May 17, 2022, the disclosure of which is incorporated herein by reference in its entirety.

### STATEMENT REGARDING FEDERALLY SPONSORED RESEARCH OR DEVELOPMENT

This invention was made with government support under Grant No. DMR1807934 awarded by the National Science Foundation. The Government has certain rights in the invention.

### TECHNICAL FIELD

The subject matter disclosed herein generally relates to zwitterion compounds and methods of making the same.

### BACKGROUND

High dielectric constant

$$( {ε}_{s} )$$

soft materials are often used for applications such as soft robotics, electronics, and batteries. The maximum energy density stored in a capacitor with an applied field E is

$$\frac{1}{2} {ε}_{0} {ε}_{s} {E}^{2} ,$$

indicating increased energy density by using high dielectric constant materials. Increasing is also essential for optimal ion transport by weakening the ion interaction energy

$$( {E}_{a} = \frac{{q}_{1} {q}_{2}}{4 {πε}_{s} r}$$

where q₁ and q₂ are the charge valence, r is the distance between ions). Soft actuators with high electrostatic pressure are more efficient in transducing the electrostatic energy into mechanical actuation, and the electrostatic pressure follows

$${P}_{ε} = {ε}_{s} {E}^{2}$$

(E is the applied field). Incorporating high dielectric constant materials is critical to improve the device performances and increase energy efficiency.

One potential way to achieve a significant dielectric constant increase for soft materials is adding zwitterions. A zwitterion is a molecule that contains an equal number of positively- and negatively-charged functional groups. Because the positive charge center and negative charge center are separated by covalent linkage, the zwitterion molecule dipole is much larger than that of polar molecules. The rarely investigated zwitterion dielectric properties are mainly because most zwitterions are crystalline solids at ambient temperature with high melting temperature Tₘ and show low dielectric constant since the molecules can barely rotate. Synthesizing high dielectric constant zwitterionic liquids is necessary to realize their potential as a high-dielectric material.

Thus, there is a need for new high dielectric constant zwitterionic liquids. There is also a need for methods of making such materials. These needs and other needs are at least partially satisfied by the present disclosure.

### SUMMARY

In accordance with the purposes of the disclosed materials, compounds, compositions, and methods, as embodied and broadly described herein, the disclosed subject matter, in one aspect, relates to compounds and compositions and methods for preparing and using such compounds and compositions.

In a further aspect, disclosed herein is a compound of Formula (I):

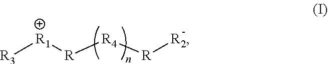

wherein
R is null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl,
R₁ is selected from a substituted or unsubstituted ammonium, imidazolium, pyrrolidinium, piperidinium, boronium, and phosphonium cation,
R₂ is selected from

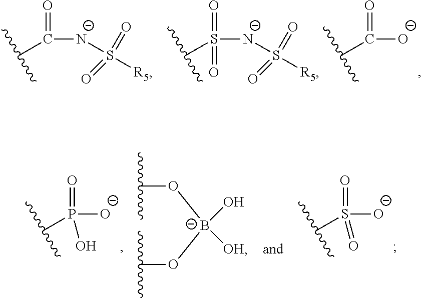

R₃ is selected from methoxyethyl, (2-methoxyethoxy)ethyl, or [2-(2-methoxyethoxy)ethoxy]ethyl, alkyl, a siloxane, a polyethylene oxide, and

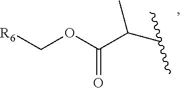

R₅ is selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, and ether; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, fluoroalkyl, and phosphonyl,
R₆ is selected from a substituted or unsubstituted crown ether, polyethylene(oxide), siloxane, and fluoroalkyl,
R₄ is selected from —CH₂—, a siloxane, a polyethylene oxide, polysulfone, polyimide, polythiophene, and —R—O—; and
n is an integer selected from 1 to 10,
wherein the compound of Formula (I) is zwitterionic and exhibits a dielectric constant higher than from about 100 to about 2000 at a temperature from about 20° C. to about 70° C.

Further, the provided herein compounds of Formula (I) are liquids at a temperature from about 20° C. to about 70° C. for at least about 1 month.

Also disclosed are aspects directed to a device comprising any of the disclosed herein compounds. In such exemplary and unlimiting aspects, the device can be a battery, an electronic device, a soft robotics device, a dielectric elastomer, a capacitor, or any combination thereof.

Still further, disclosed herein is a method of making a compound of Formula (I)

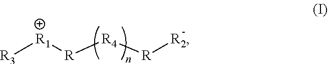

comprising:
a) reacting a compound of Formula (II)

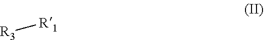

with a reagent of Formula (III)

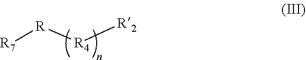

to form an intermediate compound of Formula (IV)

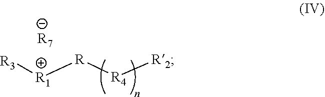

b) performing an ion-exchange of the compound of Formula (IV) to form the compound of Formula (I),
wherein
R is null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl,
R′₁ is selected from a substituted or unsubstituted amino group, imidazole group, pyrrolidine group, piperidine group, aminoborane group, and phosphine group,
R₁ is selected from substituted or unsubstituted ammonium, imidazolium, pyrrolidinium, piperidinium, boronium, organoborane, and phosphonium cation,
R′₂ is selected from

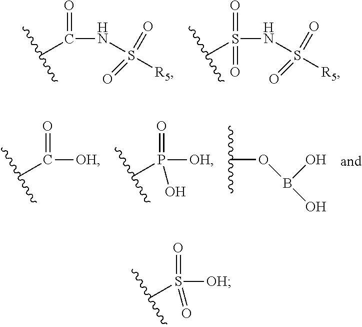

R₂ is selected from

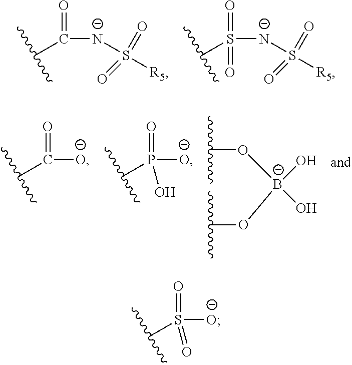

R₃ is selected from methoxyethyl, (2-methoxyethoxy)ethyl, or [2-(2-methoxyethoxy)ethoxy]ethyl, alkyl, a siloxane, a polyethylene oxide, fluoroalkyl, and

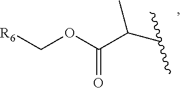

R₅ is selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, and ether; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, fluoroalkyl, and phosphonyl,
R₆ is selected from a substituted or unsubstituted crown ether, polyethylene(oxide), fluoroalkyl, and siloxane,
R₄ is selected from —CH₂—, a siloxane, a polyethylene oxide, polysulfone, polyimide, polythiophene, and —R—O—;
n is an integer selected from 1 to 10, and
R₇ is a leaving group comprising a halogen;
wherein the compound of Formula (I) is zwitterionic and exhibits a dielectric constant higher than from about 100 to about 2000 at a temperature from about 20° C. to about 70° C.

Additional advantages will be set forth in part in the description that follows and in part will be obvious from the description or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

FIG. 1 depicts a ¹H NMR spectrum of OE2Im6N (D₂O, Bruker NEO-400).

FIG. 2 depicts a ¹³C NMR spectrum of OE2Im6N. (D₂O, Bruker NEO-400).

FIG. 3 depicts a TOF-MASS spectrum of OE2Im6N. (ESI+, m/z): 416 [H]⁺, 438 [Na]⁺; calcd. 415.0.

FIG. 4 depicts a ¹H NMR spectrum of OE2Py6N. (D₂O, Bruker NEO-400).

FIG. 5 depicts a ¹³C NMR spectrum of OE2Py6N (D₂O, Bruker NEO-400).

FIG. 6 depicts a ¹H NMR spectrum of OE2Im9C (D₂O, Bruker NEO-400).

FIG. 7 depicts a temperature dependence of the dielectric constant of exemplary zwitterions. Filled symbols represent the zwitterions that can form liquids at ambient temperature. Open symbols represent the zwitterions that crystallize rapidly at ambient temperature.

FIG. 8 depicts DSC traces of zwitterionic liquids that were taken from the second heating (10 K/min heating/cooling rates) in one aspect. The heating and cooling rates are 10 K/mins. Attaching ethylene-oxide-based cation substitutes enables the formation of zwitterionic liquids at ambient temperature. OctIm3S, OE2Im5C, and OE2Im6N do not crystallize over a span of several months. OE6N3S and OE2Im4S crystallize over a span of several weeks.

FIG. 9 depicts zwitterions crystallization during the DSC measurements (second heating, 10 K/min heating/cooling rates).

FIG. 10 depicts the dielectric constant of zwitterionic liquids at 303K with respect to the number of carbons between cation and anion.

### DETAILED DESCRIPTION

The materials, compounds, compositions, articles, and methods described herein may be understood more readily by reference to the following detailed description of specific aspects of the disclosed subject matter, and the Examples included therein.

Before the present materials, compounds, compositions, kits, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

The term “comprising” and variations thereof as used herein is used synonymously with the term “including” and variations thereof and are open, non-limiting terms. Although the terms “comprising” and “including” have been used herein to describe various examples, the terms “consisting essentially of” and “consisting of” can be used in place of “comprising” and “including” to provide for more specific examples of the invention and are also disclosed. Other than in the examples, or where otherwise noted, all numbers expressing quantities of ingredients, reaction conditions, and so forth used in the specification and claims are to be understood at the very least and not as an attempt to limit the application of the doctrine of equivalents to the scope of the claims, to be construed in light of the number of significant digits and ordinary rounding approaches.

As used in the description and the appended claims, the singular forms “a,” “an,” and “the” include plural referents unless the context clearly dictates otherwise. Thus, for example, reference to “a composition” includes mixtures of two or more such compositions, reference to “the compound” includes mixtures of two or more such compounds and the like. “Optional” or “optionally” means that the subsequently described event or circumstance can or cannot occur and that the description includes instances where the event or circumstance occurs and instances where it does not.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values, inclusive of the recited values, may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It is understood that throughout this specification, the identifiers “first” and “second” are used solely to aid in distinguishing the various components and steps of the disclosed subject matter. The identifiers “first” and “second” are not intended to imply any particular order, amount, preference, or importance to the components or steps modified by these terms.

The term “complex” is used herein does not mean to describe a particular type of bonding or coordination between the compounds disclosed herein but to indicate that the compounds described herein can be associated with each other in any possible configuration.

As used herein, the term or phrase “effective,” “effective amount,” or “conditions effective to” refers to such amount or condition that is capable of performing the function or property for which an effective amount or condition is expressed. As pointed out below, the exact amount or particular condition required will vary from one aspect to another, depending on recognized variables such as the materials employed and the observed processing conditions. Thus, it is not always possible to specify an exact “effective amount” or “condition effective to.” However, it should be understood that an appropriate, effective amount will be readily determined by one of ordinary skill in the art using only routine experimentation.

### Chemical Definitions

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight, components Y, X, and Y are present at a weight ratio of 2:5 and are present in such ratio regardless of whether additional components are contained in the compound.

A weight percent (w-t. %) of a component, unless specifically stated to the contrary, is based on the total weight of the formulation or composition in which the component is included.

As used herein, the term “substituted” means that a hydrogen atom is removed and replaced by a substituent. It is contemplated to include all permissible substituents of organic compounds. As used herein, the phrase “optionally substituted” means unsubstituted or substituted. It is to be understood that substitution at a given atom is limited by valency. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein, which satisfy the valencies of the heteroatoms. This disclosure is not intended to be limited in any manner by the permissible substituents of organic compounds. Also, the terms “substitution” or “substituted with” include the implicit proviso that such substitution is in accordance with the permitted valence of the substituted atom and the substituent and that the substitution results in a stable compound, e.g., a compound that does not spontaneously undergo transformation such as by rearrangement, cyclization, elimination, etc. In still further aspects, it is understood that when the disclosure describes a group being substituted, it means that the group is substituted with one or more (i.e., 1, 2, 3, 4, or 5) groups as allowed by valence selected from alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

All compounds, and salts thereof, can be found together with other substances such as water and solvents (e.g., hydrates and solvates).

Compounds provided herein also can include tautomeric forms. Tautomeric forms result from the swapping of a single bond with an adjacent double bond together with the concomitant migration of a proton. Tautomeric forms include prototropic tautomers that are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system; for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds provided herein can also include all isotopes of atoms occurring in the intermediates or final compounds. Isotopes include those atoms having the same atomic number but different mass numbers. For example, isotopes of hydrogen include hydrogen, tritium, and deuterium.

Also provided herein are salts of the compounds described herein. It is understood that the disclosed salts can refer to derivatives of the disclosed compounds wherein the parent compound is modified by converting an existing acid or base moiety to its salt form. Examples of the salts include but are not limited to mineral or organic acid salts of basic residues such as amines; alkali or organic salts of acidic residues such as carboxylic acids; and the like. The salts of the compounds provided herein include the conventional non-toxic salts of the parent compound formed, for example, from non-toxic inorganic or organic acids. The salts of the compounds provided herein can be synthesized from the parent compound that contains a basic or acidic moiety by conventional chemical methods.

Generally, such salts can be prepared by reacting the free acid or base forms of these compounds with a stoichiometric amount of the appropriate base or acid in water or an organic solvent or in a mixture of the two. In various aspects, nonaqueous media like ether, ethyl acetate, alcohols (e.g., methanol, ethanol, isopropanol, or butanol), or acetonitrile (ACN) can be used.

In various aspects, the compounds provided herein, or salts thereof, are substantially isolated. By “substantially isolated,” it meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

As used herein, chemical structures that contain one or more stereocenters depicted with dashed and bold bonds are meant to indicate the absolute stereochemistry of the stereocenter(s) present in the chemical structure. As used herein, bonds symbolized by a simple line do not indicate a stereo-preference. Unless otherwise indicated to the contrary, chemical structures, which include one or more stereocenters, illustrated herein without indicating absolute or relative stereochemistry encompass all possible stereoisomeric forms of the compound (e.g., diastereomers and enantiomers) and mixtures thereof. Structures with a single bold or dashed line and at least one additional simple line encompass a single enantiomeric series of all possible diastereomers.

The resolution of racemic mixtures of compounds can be carried out using appropriate methods. An exemplary method includes fractional recrystallization using a chiral resolving acid that is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid, or the various optically active camphorsulfonic acids such as camphorsulfonic acid. Other resolving agents suitable for fractional crystallization methods include stereoisomerically pure forms of methylbenzylamine (e.g., S and R forms, or diastereomerically pure forms). 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent compositions can be determined by one skilled in the art.

The expressions “ambient temperature” and “room temperature” as used herein are understood in the art and refer generally to a temperature, e.g., a reaction temperature, that is about the temperature of the room in which the reaction is carried out, for example, a temperature from about 20° C. to about 30° C.

“R¹,” “R²,” “R³,” “R⁴,” etc., are used herein as generic symbols to represent various specific substituents. These symbols can be any substituents, not limited to those disclosed herein, and when they are defined to be certain substituents in one instance, they can, in another instance, be defined as some other substituents.

The chemical groups as defined and herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

At various places in the present specification, divalent linking substituents are described. It is specifically intended that each divalent linking substituent includes both the forward and backward forms of the linking substituent. For example, —NR(CR′R″)ₙ-includes both —NR(CR′R″)ₙ— and —(CR′R″)ₙNR—. Where the structure clearly requires a linking group, the Markush variables listed for that group are understood to be linking groups.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

Throughout the definitions, the term “Cₙ-Cₘ” indicates a range that includes the endpoints, wherein n and m are integers and indicate the number of carbons. Examples include, without limitation, C₁-C₄, C₁-C₆, and the like.

The term “aliphatic,” as used herein, refers to a non-aromatic hydrocarbon group and includes branched and unbranched, alkyl, alkenyl, or alkynyl groups. As used herein, the term “Cₙ-Cₘ alkyl,” employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, heptyl, octyl, nonyl, decyl, dodecyl, tetradecyl, hexadecyl, eicosyl, tetracosyl, and the like. In various aspects, the alkyl group contains from 1 to 24 carbon atoms, from 1 to 12 carbon atoms, from 1 to 10 carbon atoms, from 1 to 8 carbon atoms, from 1 to 6 carbon atoms, from 1 to 4 carbon atoms, from 1 to 3 carbon atoms, or 1 to 2 carbon atoms. The alkyl group can also be substituted or unsubstituted. Throughout the specification, “alkyl” is generally used to refer to both unsubstituted alkyl groups and substituted alkyl groups; however, substituted alkyl groups are also specifically referred to herein by identifying the specific substituent(s) on the alkyl group. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below. The term “alkyl” may refer to monovalent alkyl groups, divalent alkyl groups, or such groups with higher valencies, and is not intended to be limiting in this aspect.

For example, the term “halogenated alkyl” specifically refers to an alkyl group that is substituted with one or more halides, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below and the like.

When “alkyl” is used in one instance, and a specific term such as “alkylalcohol” is used in another, it is not meant to imply that the term “alkyl” does not also refer to specific terms such as “alkylalcohol” and the like. The term “halogenated alkyl” may refer to monovalent halogenated alkyl groups, divalent halogenated alkyl groups, or such groups with higher valencies, and is not intended to be limiting in this aspect.

As used herein, “Cₙ-Cₘ alkenyl” refers to an alkyl group having one or more double carbon-carbon bonds and having n to m carbons. Example alkenyl groups include, but are not limited to, ethenyl, n-propenyl, isopropenyl, n-butenyl, seobutenyl, and the like. In various aspects, the alkenyl moiety contains 2 to 6, 2 to 4, or 2 to 3 carbon atoms. Asymmetric structures such as (R¹R²)C═C(R³R⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, cyano, silyl, sulfo-oxo, sulfonyl, sulfone, sulfoxide, thiol, thiol, or phosphonyl, as described below. The term “alkenyl” may refer to monovalent alkenyl groups, divalent alkenyl groups, or such groups with higher valencies, and is not intended to be limiting in this aspect.

As used herein, “Cₙ-Cₘ alkynyl” refers to an alkyl group having one or more triple carbon-carbon bonds and having n to m carbons. Exemplary alkynyl groups include, but are not limited to, ethynyl, propyn-1-yl, propyn-2-yl, and the like. In various aspects, the alkynyl moiety contains 2 to 6, 2 to 4, or 2 to 3 carbon atoms. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, cyano, silyl, sulfo-oxo, sulfonyl, sulfone, sulfoxide, thiol, or phosphonyl, as described below. The term “alkynyl” may refer to monovalent alkynyl groups, divalent alkynyl groups, or such groups with higher valencies, and is not intended to be limiting in this aspect.

As used herein, the term “Cₙ-Cₘ alkylene,” employed alone or in combination with other terms, refers to a divalent alkyl linking group having n to m carbons. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl, and the like. In various aspects, the alkylene moiety contains 2 to 6, 2 to 4, 2 to 3, 1 to 6, 1 to 4, or 1 to 2 carbon atoms.

As used herein, the term “Cₙ-Cₘ alkoxy,” employed alone or in combination with other terms, refers to a group of formula —O-alkyl, wherein the alkyl group has n to m carbons. Exemplary alkoxy groups include methoxy, ethoxy, propoxy (e.g., w-propoxy and isopropoxy), tert-butoxy, and the like. In various aspects, the alkyl group has 1 to 6, 1 to 4, or 1 to 3 carbon atoms. The term “alkoxy” may refer to monovalent alkoxy groups, divalent alkoxy groups, or such groups with higher valencies, and is not intended to be limiting in this aspect.

The terms “amine” or “amino” as used herein are represented by the formula —NR¹R², where R¹ and R² can each be substitution groups as described herein, such as hydrogen, an alkyl, halogenated alkyl, alkenyl, alkynyl, aryl, heteroaryl, cycloalkyl, cycloalkenyl, heterocycloalkyl, or heterocycloalkenyl group described above. “Amido” is —C(O)NR¹R².

As used herein, the term “Cₙ-Cₘ alkylamino” refers to a group of formula —NH(alkyl), wherein the alkyl group has n to m carbon atoms. In various aspects, the alkyl group has 1 to 6, 1 to 4, or 1 to 3 carbon atoms.

As used herein, the term “Cₙ-Cₘ alkoxycarbonvl” refers to a group of formula —C(O)O-alkyl, wherein the alkyl group has n to m carbon atoms. In various aspects, the alkyl group has 1 to 6, 1 to 4, or 1 to 3 carbon atoms.

As used herein, the term “Cₙ-Cₘ alkylcarbonyl” refers to a group of formula —C(O)— alkyl, wherein the alkyl group has n to m carbon atoms. In various aspects, the alkyl group has 1 to 6, 1 to 4, or 1 to 3 carbon atoms.

As used herein, the term “Cₙ-Cₘ alkylcarbonylamino” refers to a group of formula —NHC(O)-alkyl, wherein the alkyl group has n to m carbon atoms. In various aspects, the alkyl group has 1 to 6, 1 to 4, or 1 to 3 carbon atoms.

As used herein, the term “Cₙ-Cₘ alkylsulfonylamino” refers to a group of formula —NHS(O)₂-alkyl, wherein the alkyl group has n to m carbon atoms. In various aspects, the alkyl group has 1 to 6, 1 to 4, or 1 to 3 carbon atoms.

The term “aldehyde” as used herein is represented by the formula —C(O)H.

Throughout this specification, “C(O)” or “CO” is a shorthand notation for C═O, which is also referred to herein as a “carbonyl.”

The term “carboxylic acid” as used herein is represented by the formula —C(O)OH. A “carboxylate” or “carboxyl” group as used herein is represented by the formula —C(O)O⁻.

The term “ester” as used herein is represented by the formula —OC(O)R¹ or —C(O)OR¹, where R¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula R¹OR², where R¹ and R₂ can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula R¹C(O)R², where R¹ and R² can be, independently, an alkyl, halogenated alkyl, alkenyl, alkynyl, aryl, heteroaryl, cycloalkyl, cycloalkenyl, heterocycloalkyl, or heterocycloalkenyl group described above.

As used herein, the term “aminosulfonyl” refers to a group of formula —S(O)₂NH₂.

As used herein, the term “Cₙ-Cₘ alkylaminosulfonyl” refers to a group of formula —S(O)₂NH(alkyl), wherein the alkyl group has n to m carbon atoms. In various aspects, the alkyl group has 1 to 6, 1 to 4, or 1 to 3 carbon atoms.

As used herein, the term “di(Cₙ-Cₘ alkyl)aminosulfonyl” refers to a group of formula —S(O)₂N(alkyl)₂, wherein each alkyl group independently has n to m carbon atoms.

In various aspects, each alkyl group has, independently, 1 to 6, 1 to 4, or 1 to 3 carbon atoms.

As used herein, the term “aminosulfonylamino” refers to a group of formula —NHS(O)₂NH₂.

As used herein, the term “Cₙ-Cₘ alkylaminosulfonylamino” refers to a group of formula —NHS(O)₂NH(alkyl), wherein the alkyl group has n to m carbon atoms. In various aspects, the alkyl group has 1 to 6, 1 to 4, or 1 to 3 carbon atoms.

As used herein, the term “di(Cₙ-Cₘ alkyl)aminosulfonylamino” refers to a group of formula —NHS(O)₂N(alkyl)₂, wherein each alkyl group independently has n to m carbon atoms. In various aspects, each alkyl group has, independently, 1 to 6, 1 to 4, or 1 to 3 carbon atoms.

As used herein, the term “aminocarbonylamino,” employed alone or in combination with other terms, refers to a group of formula —NHC(O)NH₂.

As used herein, the term “Cₙ-Cₘ alkylaminocarbonylamino” refers to a group of formula —NHC(O)NH(alkyl), wherein the alkyl group has n to m carbon atoms. In various aspects, the alkyl group has 1 to 6, 1 to 4, or 1 to 3 carbon atoms.

As used herein, the term “di(Cₙ-Cₘ alkyl)aminocarbonylamino” refers to a group of formula —NHC(O)N(alkyl)₂, wherein each alkyl group independently has n to m carbon atoms. In various aspects, each alkyl group has, independently, 1 to 6, 1 to 4, or 1 to 3 carbon atoms.

As used herein, the term “Cₙ-Cₘ alkylcarbamyl” refers to a group of formula —C(O)—NH(alkyl), wherein the alkyl group has n to m carbon atoms. In various aspects, the alkyl group has 1 to 6, 1 to 4, or 1 to 3 carbon atoms.

As used herein, the term “thio” refers to a group of formula —SH.

As used herein, the term “Cₙ-Cₘ alkylthio” refers to a group of formula —S-alkyl, wherein the alkyl group has n to m carbon atoms. In various aspects, the alkyl group has 1 to 6, 1 to 4, or 1 to 3 carbon atoms.

As used herein, the term “Cₙ-Cₘ alkylsulfonyl” refers to a group of formula —S(O)— alkyl, wherein the alkyl group has n to m carbon atoms. In various aspects, the alkyl group has 1 to 6, 1 to 4, or 1 to 3 carbon atoms.

As used herein, the term “Cₙ-Cₘ alkylsulfonyl” refers to a group of formula —S(O)₂-alkyl, wherein the alkyl group has n to m carbon atoms. In various aspects, the alkyl group has 1 to 6, 1 to 4, or 1 to 3 carbon atoms.

As used herein, the term “carbamyl” refers to a group of formula —C(O)NH₂.

As used herein, the term “carbonyl,” employed alone or in combination with other terms, refers to a —C(═O)— group, which may also be written as C(O).

As used herein, the term “carboxy” refers to a group of formula —C(O)OH.

As used herein, the term “(Cₙ-Cₘ)(Cₙ-Cₘ)amino” refers to a group of formula —N(alkyl)₂, wherein the two alkyl groups each has, independently, n to m carbon atoms. In various aspects, each alkyl group independently has 1 to 6, 1 to 4, or 1 to 3 carbon atoms.

As used herein, the term “di(Cₙ-Cₘ-alkyl)carbamyl” refers to a group of formula —C(O)N(alkyl)₂, wherein the two alkyl groups each have, independently, n to m carbon atoms. In various aspects, each alkyl group independently has 1 to 6, 1 to 4, or 1 to 3 carbon atoms.

As used herein, “halogen” refers to F, Cl, Br, or I.

The term “hydroxyl” as used herein is represented by the formula —OH.

The term “cyano” as used herein is represented by the formula —CN.

The term “nitro” as used herein is represented by the formula —NO₂.

The term “phosphonyl” is used herein to refer to the phospho-oxo group represented by the formula —P(O)(OR¹)₂, where R¹ can be absent, hydrogen, an alkyl, alkenyl, alkynyl, aryl, heteroaryl, cycloalkyl, or cycloalkenyl.

The term “silyl” as used herein is represented by the formula —SiR¹R²R³, where R¹, R², and R³ can be, independently, hydrogen, alkyl, halogenated alkyl, alkoxy, alkenyl, alkynyl, aryl, heteroaryl, cycloalkyl, cycloalkenyl, heterocycloalkyl, or heterocycloalkenyl group described above.

The term “sulfonyl” is used herein to refer to the sulfo-oxo group represented by the formula —S(O)₂R¹, where R¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH—.

As used herein, “Cₙ-Cₘ haloalkoxy” refers to a group of formula —O-haloalkyl having n to m carbon atoms. An example haloalkoxy group is OCF₃. In various aspects, the haloalkoxy group is fluorinated only. In various aspects, the alkyl group has 1 to 6, 1 to 4, or 1 to 3 carbon atoms.

As used herein, the term “Cₙ-Cₘ haloalkyl,” employed alone or in combination with other terms, refers to an alkyl group having from one halogen atom to 2s+1 halogen atoms, which may be the same or different, where “s” is the number of carbon atoms in the alkyl group, wherein the alkyl group has n to m carbon atoms. In various aspects, the haloalkyl group is fluorinated only. In various aspects, the alkyl group has 1 to 6, 1 to 4, or 1 to 3 carbon atoms.

As used herein, the term “amine base” refers to a mono-substituted amino group (i.e., primary amine base), di-substituted amino group (i.e., secondary amine base), or a tri-substituted amine group (i.e., tertiary amine base). Exemplary mono-substituted amine bases include methylamine, ethylamine, propylamine, butylamine, and the like. Examples of di-substituted amine bases include dimethylamine, diethylamine, dipropylamine, dibutylamine, pyrrolidine, piperidine, azepane, morpholine, and the like. In various aspects, the tertiary amine has the formula N(R′)₃, wherein each R¹ is independently C₁-C₆ alkyl, 3-10 member cycloalkyl, 4-10 membered heterocycloalkyl, 1-10 membered heteroaryl, and 5-10 membered aryl, wherein the 3-10 member cycloalkyl, 4-10 membered heterocycloalkyl, 1-10 membered heteroaryl, and 5-10 membered aryl is optionally substituted by 1, 2, 3, 4, 5, or 6 C₁₋₆ alkyl groups. Exemplary tertiary amine bases include trimethylamine, triethylamine, tripropylamine, triisopropylamine, tributylamine, tri-tert-butylamine, N,N-dimethylethanamine, N-ethyl-N-methylpropan-2-amine, N-ethyl-N-isopropylpropan-2-amine, morpholine, N-methylmorpholine, and the like. In various aspects, the term “tertiary amine base” refers to a group of formula N(R)₃, wherein each R is independently a linear or branched C₁₋₆ alkyl group.

As used herein, “cycloalkyl” refers to non-aromatic cyclic hydrocarbons, including cyclized alkyl and/or alkenyl groups. Cycloalkyl groups can include mono- or polycyclic (e.g., having 2, 3, or 4 fused rings) groups and spirocycles. Cycloalkyl groups can have 3, 4, 5, 6, 7, 8, 9, or 10 ring-forming carbons (C₃₋₁₀). Ring-forming carbon atoms of a cycloalkyl group can be optionally substituted by oxo or sulfido (e.g., C(O) or C(S)). Cycloalkyl groups also include cycloalkylidenes. Exemplary cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbomyl, norpinyl, norcarnyl, and the like. In various aspects, cycloalkyl is cyclopropyl, cyclobutyl, cyclopentyl, cyclohexyl, cyclopentyl, or adamantyl. In various aspects, the cycloalkyl has 6-10 ring-forming carbon atoms. In various aspects, cycloalkyl is cyclohexyl or adamantyl. Also included in the definition of cycloalkyl are moieties that have one or more aromatic rings fused (i.e., having a bond in common with) to the cycloalkyl ring, for example, benzo or thienyl derivatives of cyclopentane, cyclohexane, and the like. A cycloalkyl group containing a fused aromatic ring can be attached through any ring-forming atom, including a ring-forming atom of the fused aromatic ring. The term “cycloalkyl” may refer to monovalent cycloalkyl groups, divalent cycloalkyl groups, or such groups with higher valencies, and is not intended to be limiting in this aspect.

As used herein, “heterocycloalkyl” refers to non-aromatic monocyclic or polycyclic heterocycles having one or more ring-forming heteroatoms selected from O, N, or S. Included in heterocycloalkyl are monocyclic 4-, 5-, 6-, and 7-membered heterocycloalkyl groups. Heterocycloalkyl groups can also include spirocycles. Exemplary heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido (e.g., C(O), S(O), C(S), or S(O)₂, etc.). The heterocycloalkyl group can be attached through a ring-forming carbon atom or a ring-forming heteroatom. In various aspects, the heterocycloalkyl group contains 0 to 3 double bonds. In various aspects, the heterocycloalkyl group contains 0 to 2 double bonds. Also included in the definition of heterocycloalkyl are moieties that have one or more aromatic rings fused (i.e., having a bond in common with) to the cycloalkyl ring, for example, benzo or thienyl derivatives of piperidine, morpholine, azepine, etc. A heterocycloalkyl group containing a fused aromatic ring can be attached through any ring-forming atom, including a ring-forming atom of the fused aromatic ring. In various aspects, the heterocycloalkyl has 4-10, 4-7, or 4-6 ring atoms with 1 or 2 heteroatoms independently selected from nitrogen, oxygen, or sulfur and having one or more oxidized ring members. The term “heterocycloalkyl” may refer to monovalent heterocycloalkyl groups, divalent heterocycloalkyl groups, or such groups with higher valencies, and is not intended to be limiting in this aspect.

The term “cycloalkenyl,” as used herein, is a non-aromatic carbon-based ring composed of at least three carbon atoms and containing at least one double bond, i.e., C═C. Examples of cycloalkenyl groups include, but are not limited to cyclopropenyl, cyclobutenyl, cyclopentenyl, cyclopentadienyl, cyclohexenyl, cyclohexadienyl, and the like.

The term “heterocycloalkenyl” is a type of cycloalkenyl group as defined above and is included within the meaning of the term “cycloalkenyl,” where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, cyano, silyl, sulfo-oxo, sulfonyl, sulfone, sulfoxide, thiol, or phosphonyl, as described herein. The term “cycloalkenyl” may refer to monovalent cycloalkenyl groups, divalent cycloalkenyl groups, or such groups with higher valencies, and is not intended to be limiting in this aspect.

The term “cyclic group” is used herein to refer to either aryl groups or non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, one or more aryl groups, and one or more non-aryl groups.

As used herein, the term “aryl,” employed alone or in combination with other terms, refers to an aromatic hydrocarbon group, which may be monocyclic or polycyclic (e.g., having 2, 3, or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In various aspects, aryl groups have from 6 to about 20 carbon atoms, from 6 to about 15 carbon atoms, or from 6 to about 10 carbon atoms. In various aspects, the aryl group is a substituted or unsubstituted phenyl. The term “aryl” may refer to monovalent aryl groups, divalent aryl groups, or such groups with higher valencies, and is not intended to be limiting in this aspect.

As used herein, “heteroaryl” refers to a monocyclic or polycyclic aromatic heterocycle having at least one heteroatom ring member selected from sulfur, oxygen, phosphorus, and nitrogen. In various aspects, the heteroaryl ring has 1, 2, 3, or 4 heteroatom ring members independently selected from nitrogen, sulfur, and oxygen. In various aspects, any ring-forming N in a heteroaryl moiety can be an N-oxide. In various aspects, the heteroaryl has 5-10 ring atoms and 1, 2, 3, or 4 heteroatom ring members independently selected from nitrogen, sulfur, and oxygen. In various aspects, the heteroaryl has 5-6 ring atoms and 1 or 2 heteroatom ring members independently selected from nitrogen, sulfur, and oxygen. In various aspects, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl, and pyridazinyl. The term “heteroaryl” may refer to monovalent heteroaryl groups, divalent heteroaryl groups, or such groups with higher valencies, and is not intended to be limiting in this aspect.

The aryl or heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, cyano, silyl, sulfo-oxo, sulfonyl, sulfone, sulfoxide, thiol, or phosphonyl, as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

In certain places, the definitions or aspects refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

As used herein, the term “electron withdrawing group” (EWG), employed alone or in combination with other terms, refers to an atom or group of atoms substituted onto a π-system (e.g., substituted onto an aryl or heteroaryl ring) that draws electron density away from the π-system through induction (e.g., withdrawing electron density about a σ-bond) or resonance (e.g., withdrawing electron density about a π-bond or π-system). Example electron withdrawing groups include, but are not limited to, halo groups (e.g., fluoro, chloro, bromo, iodo), nitriles (e.g., —CN), carbonyl groups (e.g., aldehydes, ketones, carboxylic acids, acid chlorides, esters, and the like), nitro groups (e.g., —NO₂), haloalkyl groups (e.g., —CH₂F, —CHF₂, —CF₃, and the like), alkenyl groups (e.g., vinyl), alkynyl groups (e.g., ethynyl), sulfonyl groups (e.g., S(O)R, S(O)₂R), sulfonate groups (e.g., —SO₃H), and sulfonamide groups (e.g., S(O)N(R)₂, S(O)₂N(R)═). In various aspects, the electron withdrawing group is selected from the group consisting of halo, C₂-C₆ alkenyl, C₂-C₆ alkynyl, C₁-C₃ haloalkyl, CN, NO₂, C(═O)OR^{a1}, C(═O)R^{b1}, C(═O)NR^{c1}R^{d1}, C(═O)SR^{e1}, —NR^{c1}S(O)R^{e1}, —NR^{c1}S(O)₂R^{e1}, S(═O)R^{e1}, S(═O)₂R^{e1}, S(═O)NR^{c1}R^{d1}, S(═O)₂NR^{c1}R^{d1}, and P(O)(OR^{a1})₂. In various aspects, the electron withdrawing group is selected from the group consisting of C(═O)OR^{a1}, C(═O)R^{b1}, C(═O)NR^{c1}R^{d1}, C(═O)SR^{e1}, S(═O)R^{e1}, S(═O)₂R^{e1}, S(═O)NR^{c1}R^{d1}, and S(═O)₂NR^{c1}R^{d1}. In various aspects, the electron withdrawing group is C(═O)OR^{a1}. In various aspects, the electron withdrawing group is C(═O)OR^{a1}, wherein Rai, R^{b1}, R^{c1}, R^{d1}, and R^{e1} are independently selected at each occurrence from alkyl, alkenyl, alkynyl, cycloalkyl, cycloalkenyl, heterocycloalkyl, aryl, or heteroaryl, each of which Rai, R^{b1}, R^{c1}, R^{d1}, or R^{e1} may be optionally substituted with one or more substituents as described herein.

“R¹,” “R²,” “R³,” “Rⁿ,” etc., where n is some integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an amine group, an alkyl group, a halide, and the like. Depending upon the groups that are selected, a first group can be incorporated within the second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

The term “protecting groups” as used herein refers to any molecular framework that can temporarily mask a specific functional group to block its reactivity under reaction conditions when modifications are needed elsewhere in the molecule. It is understood that the protecting groups can comprise any molecular framework that can be selectively introduced, stable, resistant to reagents employed in subsequent reaction steps in which the group is masked (protected) is desired to remain deactivated (protected). It is further understood that the protecting groups used herein can be selectively removed under mild conditions when their protection is no longer required. In some exemplary and non-limiting aspects, the protecting groups can comprise ethers, silyls, acetals, ketals, esters, carbamates, and the like. In still further exemplary aspects described herein, the protecting group can comprise a benzyl group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Dashed lines in a chemical structure are used to indicate that a bond may be present or absent or that it may be a delocalized bond between the indicated atoms. For example, —(C═O)NH₂ is attached through the carbon of the keto (C═O) group.

“Leaving group,” as used herein, refers to a molecule or a molecular fragment (e.g., an anion) that is displaced in a chemical reaction as stable species taking with it the bonding electrons. Examples of leaving groups include an arylsulfonyloxy group or an alkylsulfonyloxy group, such as a mesylate or a tosylate group. Common anionic leaving groups also include halides such as Cl—, Br—, and I—.

As used herein, the term “composition” is intended to encompass a product comprising the specified ingredients in the specified amounts, as well as any product which results, directly or indirectly, from a combination of the specified ingredients in the specified amounts.

As used herein, substantially pure means sufficiently homogeneous to appear free of readily detectable impurities as determined by standard methods of analysis, such as thin-layer chromatography (TLC), nuclear magnetic resonance (NMR), gel electrophoresis, high-performance liquid chromatography (HPLC), and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modem methods for purification of the compounds to produce substantially chemically pure compounds are known to those of skill in the art. A substantially chemically pure compound can, however, be a mixture of stereoisomers.

Preparation of the compounds described herein can involve a reaction in the presence of an acid or a base. Example acids can be inorganic or organic acids and include, but are not limited to, strong and weak acids. Example acids include but are not limited to hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Example weak acids include, but are not limited to, acetic acid, propionic acid, butanoic acid, benzoic acid, tartaric acid, pentanoic acid, hexanoic acid, heptanoic acid, octanoic acid, nonanoic acid, and decanoic acid. Examples include, without limitation, lithium hydroxide, sodium hydroxide, potassium hydroxide, lithium carbonate, sodium carbonate, potassium carbonate, sodium bicarbonate, and amine bases. Example strong bases include, but are not limited to, hydroxide, alkoxides, metal amides, metal hydrides, metal dialkylamides, and arylamines, wherein; alkoxides include lithium, sodium, and potassium salts of methyl, ethyl, and t-butyl oxides; metal amides include sodium amide, potassium amide, and lithium amide; metal hydrides include sodium hydride, potassium hydride, and lithium hydride; and metal dialkylamides include lithium, sodium, and potassium salts of methyl, ethyl, n-propyl, iso-propyl, n-butyl, t-butyl, trimethylsilyl, and cyclohexyl substituted amides (e.g., lithium N-isopropylcyclohexylamide).

The following abbreviations may be used herein: AcOH (acetic acid); aq. (aqueous); atm. (atmosphere(s)); Br₂ (bromine); Bn (benzyl); calc. (calculated); d (doublet); dd (doublet of doublets); DCM (dichloromethane); DMF (N,N-dimethylformamide); Et (ethyl); Et₂O (diethyl ether); EtOAc (ethyl acetate); EtOH (ethanol); EWG (electron withdrawing group); g (gram(s)); h (hour(s)); HCl (hydrochloric acid/hydrogen chloride); HPLC (high performance liquid chromatography); H₂SO₄ (sulfuric acid); Hz (hertz); (iodine); IPA (isopropyl alcohol); J (coupling constant); KOH (potassium hydroxide); K₃PO₄ (potassium phosphate); LCMS (liquid chromatography—mass spectrometry); GC (gas chromatography), LilCA (lithium N-isopropylcyclohexylamide); m (multiplet); M (molar); MS (Mass spectrometry); Me (methyl); MeCN (acetonitrile); MeOH (methanol); mg (milligram(s)); min. (minutes(s)); mL (milliliter(s)); mmol (millimole(s)); N (normal); NaBH₄CN (sodium cyanoborohydride); NHP (N-heterocyclic phosphine); NHP—Cl (N-heterocyclic phosphine chloride); Na₂CO₃ (sodium carbonate); NaHCO₃ (sodium bicarbonate); NaOH (sodium hydroxide); Na₂SO₄ (sodium sulfate); nM (nanomolar); NMR (nuclear magnetic resonance spectroscopy); PCb (trichlorophosphine); PMP (4-methoxyphenyl); RP-HPLC (reverse phase high performance liquid chromatography); t (triplet or tertiary); t-Bu (tert-butyl); TEA (triethylamine); TFA (trifluoroacetic acid); THF (tetrahydrofuran); TLC (thin layer chromatography); μg (microgram(s)); μL (microliter(s)); μM (micromolar); wt % (weight percent).

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in detail to specific aspects of the disclosed materials, compounds, compositions, articles, and methods, examples of which are illustrated in the accompanying Examples.

### Compounds

Disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, suppose a composition is disclosed, and a number of modifications that can be made to a number of components of the composition are discussed. In that case, each and every combination and permutation that are possible are specifically contemplated unless specifically indicated to the contrary. Thus, if a class of components A, B, and C are disclosed and a class of components D, E, and F and an example of a combination composition A-D are disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from the disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure, including, but not limited to, steps in methods of making and using the disclosed compositions. Thus, if there are a variety of additional steps that can be performed, it is understood that each of these additional steps can be performed with any specific aspect or combination of aspects of the disclosed methods and that each such combination is specifically contemplated and should be considered disclosed.

In certain aspects disclosed herein are zwitterion compounds. In certain aspects, the zwitterion compounds can be referred to as zwitterion ionic liquids. It is understood that these two terms can be used interchangeably.

It is understood that the zwitterion is a compound wherein a cation and an anion coexist in one molecule. In the zwitterion compounds, both of a cation and an anion are fixed in a molecule and thus are repressed from movement along a potential gradient and fixed between both electrodes. Therefore, a high-speed ion-conducting path, which allows only desired ion to move, can be formed. It is advantageous that there is no deterioration of electric characteristics caused by an uneven distribution of electric charge.

In still further aspects, disclosed herein are compounds of Formula (I):

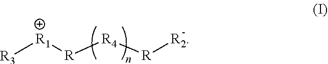

In such aspects, R can be any linkage chain. For example, and without limitations, R can be null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, phosphonyl, and the like.

In still further aspects, R₁ can be any onium cation suitable for the desired application. In such aspects, the onium cation is not especially limited, so long as it is a cation having at least one organic group formed by coordinating a cation type atomic group to a compound containing an element having an isolated electron pair such as nitrogen, sulfur, oxygen, phosphorus, selenium, tin, iodine, and antimony. Examples of an organic onium ion that can be used in the present invention include symmetric ammonium cations such as a tetramethylammonium cation, tetraethylammonium cation and tetrapropylammonium cation; ammonium cations, in which the number of carbon atoms of the shortest substituent is not less than 50% and less than 100% of the number of carbon atoms of the longest substituent (hereinafter, may be referred to as pseudo symmetric), such as an ethyltrimethylammonium cation, vinyltrimethylammonium cation, triethylmethylammonium cation, triethylpropylammonium cation, diethyldimethylammonium cation, tributylethylammonium cation, triethylisopropylammonium cation, N,N-dimethylpyrrolidinium cation, N-methyl-N-ethylpyrrolidinium cation and triethylmethoxymethylammonium cation; asymmetric ammonium cations such as a trimethylpropylammonium cation, trimethylisopropylammonium cation, butyltrimethylammonium cation, allyltrimethylammonium cation, hexyltrimethylammonium cation, octyltrimethylammonium cation, dodecyltrimethylammonium cation, triethylmethoxyethoxymethylammonium cation and dimethyldipropylammonium cation; divalent ammonium cations such as a hexamethonium cation; symmetric imidazolium cations such as a 1,3-dimethylimidazolium cation, 1,3-diethylimidazolium cation, 1,3-dipropylimidazolium cation and 1,3-dibutylimidazolium cation; asymmetric imidazolium cations such as an 1-ethyl-3-methylimidazolium cation, 1-methyl-3-propylimidazolium cation, 1-isopropyl-3-propylimidazolium cation and 1-tert-butyl-3-isopropylimidazolium cation; pyridinium cations such as an N-ethylpyridinium cation and N-butylpyridinium cation; symmetric sulfonium cations such as a trimethylsulfonium cation, triethylsulfonium cation and tributylsulfonium cation; pseudo symmetric sulfonium cations such as a diethylmethylsulfonium cation; asynmmetric sulfonium cations such as a dimethylpropylsulfonium and dimethylhexylsulfonium; symmetric phosphonium cations such as a tetramethylphosphonium cation, tetraethylphosphonium cation, tetrapropylphosphonium cation, tetrabutylphosphonium cation, tetraoctylphosphonium cation and tetraphenylphosphonium cation; pseudo symmetric phosphonium cations such as a trimethylethylphosphonium cation and triethylmethylphosphonium cation; asymmetric phosphonium cations such as a hexyltrimethylphosphonium cation and trimethyloctylphosphonium cation; and the like.

Yet, in still further aspects, R₁ is selected from a substituted or unsubstituted ammonium, imidazolium, pyrrolidinium, piperidinium, boronium, and phosphonium cation. It is understood that any known in the art substitutions can be present on the onium cation.

In still further aspects, R₃ can be represented by a hydrogen atom, an alkyl group having 1 to 18 carbon atoms, an aryl group, a heterocyclic group, an aralkyl group, alkene group, arylene group, alkoxy group, aryloxy group, siloxane group, polyether group, fluoroalkyl, and the like. R₃ can also be substituted or unsubstituted. The alkyl group, if present, can have 1 to 18 carbon atoms includes straight or branched-chain alkyl groups having 1 to 18 carbon atoms, such as methyl group, ethyl group, n-propyl group, isopropyl group, n-butyl group, isobutyl group, sec-butyl group, tert-butyl group, pentyl group, hexyl group, heptyl group, octyl group, nonyl group, and decyl group. In aspects where the aryl group is present, it can include a phenyl group, naphthyl group, toluyl group, and xylyl group. In still further aspects, the heterocyclic group can comprise a pyridyl group, thienyl group, imidazolyl group, pyrazolyl group, oxazolyl group, isooxazolyl group, pyrrolidinyl group, piperazinyl group, and morpholinyl group. In yet other aspects, the alkylene group can comprise 1 to 18 carbon atoms and include a straight or branched-chain group having 1 to 18 carbon atoms such as methylene group, ethylene group, propylene group, butylene group, pentylene group, hexylene group, heptylene group, octylene group, nonylene group, and decylene group. In yet still further aspects, the arylene group can comprise a phenylene group, naphthylene group, toluylene group, and xylylene group. In yet still further aspects, the heterocyclic group includes a pyridylene group, thienylene group, imidazolylene group, pyrazolylene group, oxazolylene group, pyrrolidinylene group, piperazinylene group, and morpholinylene group.

In still further aspects, R₃ can comprise a substituent in the structure thereof. Such substituent includes halogen atoms (fluorine atom, chlorine atom, bromine atom, and iodine atom), hydroxyl group, alkoxy group (methoxy group, ethoxy group, propoxy group, butoxy group, and the like), carboxyl group, acetyl group, propanoyl group, thiol group, alkylthio group (methylthio group, ethylthio group, propylthio group, butylthio group and the like), amino group, alkylamino group, and dialkylamino group and the like.

In still further aspects, R₃ is selected from methoxyethyl, (2-methoxyethoxy)ethyl, or [2-(2-methoxyethoxy)ethoxy]ethyl, alkyl, a siloxane, a polyethylene oxide, and

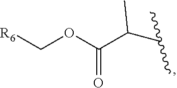

where R₆ can be a substituted or unsubstituted crown ether, polyethylene(oxide), fluoroalkyl, or siloxane.

In still further aspects, the substituted or unsubstituted crown ether, if present, can be selected from

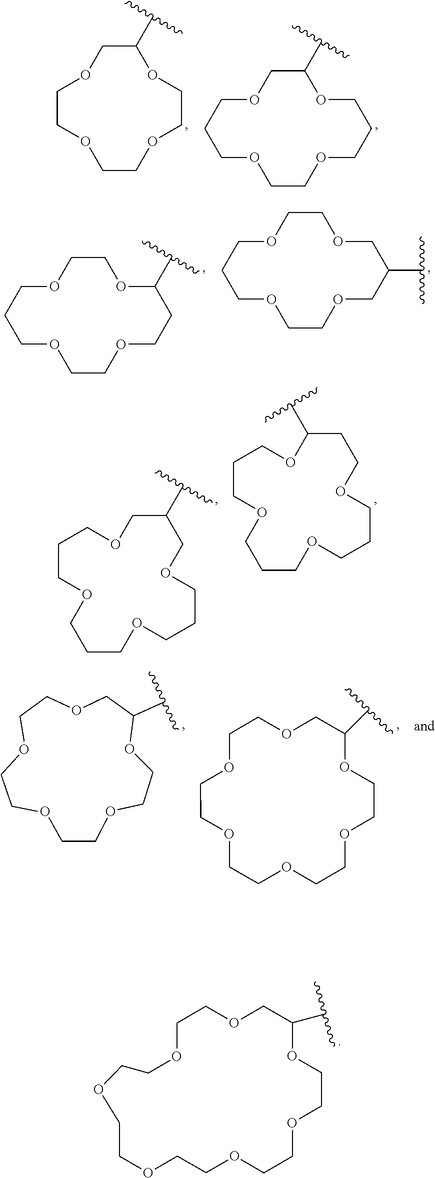

In still further aspects, R₂ is an anion that can be selected from

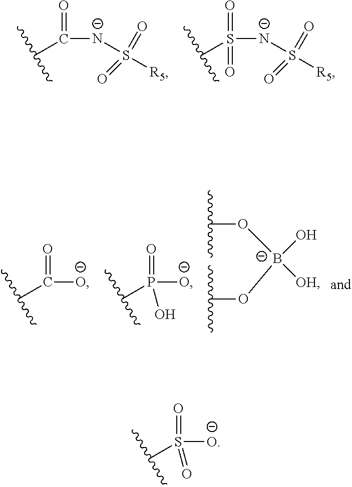

In such exemplary and unlimiting aspects, R₅ can be selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, ether, and fluoroalkyl; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, fluoroalkyl, phosphonyl, and the like.

In still further exemplary and unlimiting aspects, R₅ can be a substituted or unsubstituted monovalent hydrocarbon group or a fluoroalkyl group (—(CF₂)ₙF) having 1 to 15 carbon atoms. In certain aspects, R₅ can be a halogen atom or a halogenated hydrocarbon. For example, and without limitations, R₅ can be a fluorine-substituted hydrocarbon group. In such exemplary and unlimiting aspects, the fluorine-substituted hydrocarbon group can comprise fluoroalkyl groups such as trifluoromethyl group, pentafluoroethyl group, heptafluoropropyl group, nonafluorobutyl group, heptafluoroisopropyl group, nonafluoroisobutyl group, 2,2,2-trifluoroethyl group, and 1,1-difluoroethyl group; fluoroaryl groups such as pentafluorophenyl group and 2,4,6-trifluorophenyl group; and fluoroaralkyl groups such as heptafluorobenzyl group and 1,1-difluorobenzyl group. In yet still further aspects, R₅ can comprise a straight or branched-chain perfluoroalkyl group having 1 to 15 carbon atoms, a perfluorophenyl group, and a perfluoroaralkyl group having 7 to 9 carbon atoms.

In still further aspects, R₅ can be (CF₂)ₙCF₃ or (CH₂)ₙCF₃, wherein n is from 1 to 15, including 1, 2, 3, 4, 5, 6, 7, 8, 9, 10, 11, 12, 13, and 14. In yet still further aspects, R₅ can be —CF₃, —(CH₂CH₂)CF₃, or —(CF₂)₇CF₃.

In still further aspects, R₄ represents a bridge between the cation and anion components of the zwitterion compound. In such aspects, R₄ can be selected from —CH₂—, a siloxane, a polyethylene oxide, polysulfone, polyimide, polythiophene, and —R—O—.

In still further aspects, n is an integer selected from from 1 to 30, including exemplary values of 2, 3, 4, 5, 6, 7, 8, 9, 10, 11, 12, 13, 14, 15, 16, 17, 18, 19, 20, 21, 22, 23, 24, 25, 26, 27, 28, and 29.

In yet still further aspects, the zwitterionic compounds disclosed herein exhibit a dielectric constant higher than from about 100 to about 2000, including exemplary values of about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 450, about 500, about 550, about 600, about 650, about 700, about 750, about 800, about 1000, about 1250, about 1500, and about 1750 at a temperature from about 20° C. to about 70° C., including exemplary values of about 25° C., about 30° C., about 35° C., about 40° C., about 45° C., about 50° C., about 55° C., about 60° C., and about 65° C.

In still further aspects, the compounds disclosed herein can be liquids at a temperature from about 20° C. to about 70° C., including exemplary values of about 25° C., about 30° C., about 35° C., about 40° C., about 45° C., about 50° C., about 55° C., about 60° C., and about 65° C. for at least about 1 day, at least about 2 days, at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 12 months, at least about 2 years, at least about 5 years, or even at least about 10 years. In yet still further aspects, the compounds disclosed herein are substantially free of crystallization for about 1 to about 12 months, including exemplary values of about 2 months, about 3 months, about 4 months, about 5 months, about 6 months, about 7 months, about 8 months, about 9 months, about 10 months, and about 11 months at a temperature from about 20° C. to about 70° C., including exemplary values of about 25° C., about 30° C., about 35° C., about 40° C., about 45° C., about 50° C., about 55° C., about 60° C., and about 65° C. In yet still further aspects, the compounds disclosed herein are substantially free of crystallization for about 2 years, 5 years, or even 10 years at a temperature from about 20° C. to about 70° C., including exemplary values of about 25° C., about 30° C., about 35° C., about 40° C., about 45° C., about 50° C., about 55° C., about 60° C., and about 65° C.

In still further aspects, the compounds of Formula I of the current disclosure can comprise at least one or more of

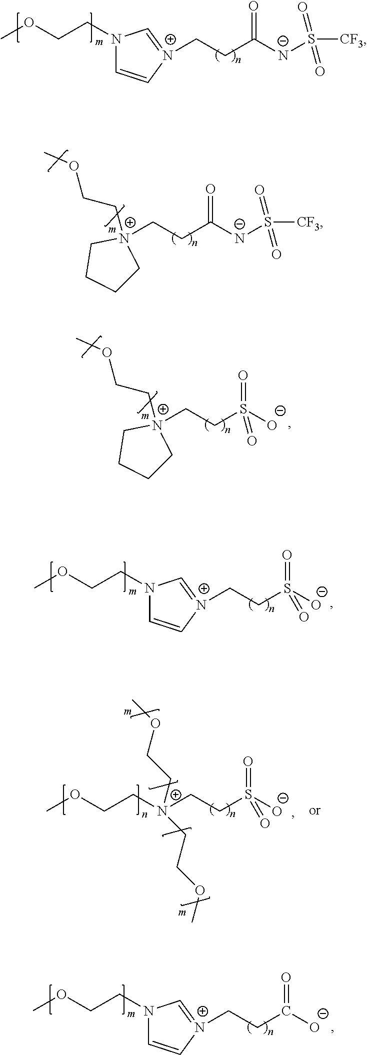

wherein m is from 1 to 6, including exemplary values of 2, 3, 4, and 5, and n is from 1 to 15, including exemplary values of 2, 3, 4, 5, 6, 7, 8, 9, 10, 11, 12, 13, and 14.

In still further aspects, the compound of Formula I comprises

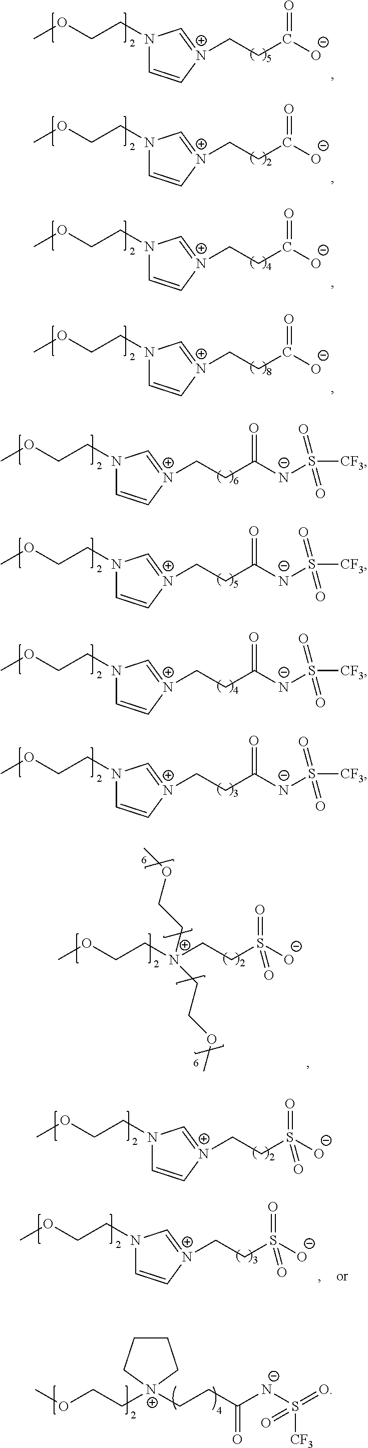

Also disclosed herein are devices that can comprise any of the disclosed above compounds. For example, and without limitations, the devices can comprise a battery, an electronic device, a soft robotics device, dielectric elastomers, capacitors, or any combination thereof

### Methods

In certain aspects, also disclosed herein are methods of making a compound of Formula (I)

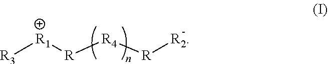

In such aspects, the method comprises
a) reacting a compound of Formula (II)

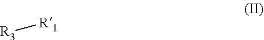

with a reagent of Formula (III)

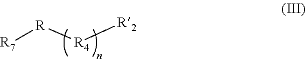

to form an intermediate compound of Formula (IV)

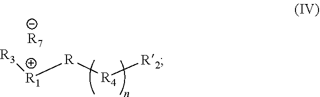

b) performing an ion-exchange of the compound of Formula (IV) to form the compound of Formula (I).

It is understood that any of the disclosed above R₃, R₄, R, or R₁ groups can be utilized.

In still further aspects, R is null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, phosphonyl, and the like.

Yet in still further aspects, R′₁ is selected from a substituted or unsubstituted amino group, imidazole group, pyrrolidine group, piperidine group, aminoborane group, and phosphine group. In yet still further aspects, R₁ is selected from substituted or unsubstituted ammonium, imidazolium, pyrrolidinium, piperidinium, boronium, and phosphonium cation.

In yet still further aspects, R′₂ is selected from

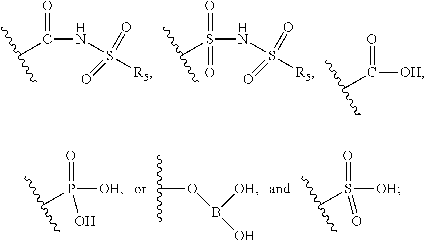

while in other aspects, R₂ is selected from

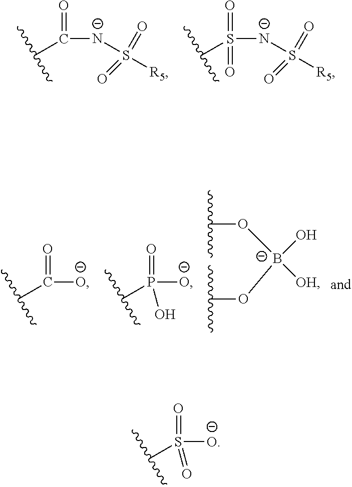

In still further aspects, R₃ is selected from methoxyethyl, (2-methoxyethoxy)ethyl, or [2-(2-methoxyethoxy)ethoxy]ethyl, alkyl, a siloxane, a polyethylene oxide, fluoroalkyl, and

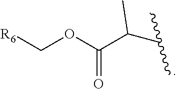

In yet still further aspects, R₅ is C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, ether, or fluoroalkyl; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, phosphonyl, and the like. In still further exemplary and unlimiting aspects, still further aspects, R₅ can be (CF₂)ₙCF₃ or (CH₂)ₙCF₃, wherein n is from 1 to 15, including 2, 3, 4, 5, 6, 7, 8, 9, 10, 11, 12, 13, and 14. In yet still further aspects, R₅ can be —CF₃, —(CH₂CH₂)CF₃, or —(CF₂)₇CF₃.

In some aspects, R₆ is a substituted or unsubstituted crown ether, polyethylene(oxide), fluoroalkyl, or siloxane.

Yet, in other aspects, R₄ is selected from —CH₂—, a siloxane, a polyethylene oxide, polysulfone, polyimide, polythiophene, and —R—O—.

In the methods disclosed herein, n can be and integer from 1 to 15 including exemplary values of 2, 3, 4, 5, 6, 7, 8, 9, 10, 11, 12, 13, and 14.

In still further aspects, R₇ is a leaving group comprising a halogen.

In some aspects, the ion exchange can be performed by any known in the art method that is suitable for the desired application. In yet still further aspects, the ion exchange step can be performed at room temperature in deionized water. In some aspects, the ion exchange is performed with an ion exchange resin. In some aspects, the ion exchange resin could be any basic resin having OH— groups. For example, and without limitations, the ion exchange resin can be Amberlyst® A26.

In still further aspects, the reagent of Formula (III) is formed by reacting compound of Formula (V)

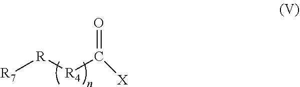

with a compound of Formula (VI)

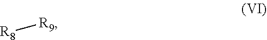

in the presence of a base,
wherein X is a halogen,
R₈ is H, C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, or C₆-C₁₄ aryloxy; wherein R⁸ is optionally substituted with one or more groups selected from C₁₋₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, phosphonyl, and the like, and
and R₉ is independently selected from —NH₂, —OH, and —COO⁻.

In yet further aspects, the base can be any base suitable for the desired purpose. In some exemplary and unlimiting aspects, the base can comprise triethylamine, methylimidazole, ethyl imidazole, N,N-diisopropylethylamine, 4-dimethylaminopyridine, or pyridine.

In still further aspects, the reaction can be conducted in the presence of a solvent. In such exemplary and unlimiting aspects, the solvent can comprise acetone, acetonitrile, tetrahydrofuran, toluene, benzene, or any combination thereof.

Any of the disclosed above zwitterion compounds can be formed by the described methods.

In yet still further aspects, the compound of Formula (I) can be formed with a yield from about 10% to 100%, including exemplary values of about 20%, about 30%, about 40%, about 50%, about 60%, about 70%, about 80%, about 90%, about 95%, and about 99%. In still further aspects, the yield for forming of the compound of Formula (I) can be greater than about 10%, greater than about 20%, greater than about 30%, greater than about 40%, greater than about 50%, greater than about 60%, greater than about 70%, greater than about 80%, greater than about 90%, greater than about 95%, or greater than about 99%.

The examples below are intended to further illustrate certain aspects of the methods and compositions described herein and are not intended to limit the scope of the claims.

### EXAMPLES

The following examples are set forth below to illustrate the methods and results according to the disclosed subject matter. These examples are not intended to be inclusive of all aspects of the subject matter disclosed herein but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions, that can be used to optimize the product purity and yield obtained from the described process. Only reasonable and routine experimentation will be required to optimize such process conditions.

Here, disclosed is a synthesis method that results in zwitterionic liquids with a very high dielectric constant, for example such as 50 to 2000, including the numbers disclosed above at temperatures between 20° C. to 200° C. Notably, the disclosed synthesis method is more advantageous than previously reported methods because 1) accessibility to various cation-anion linkage lengths (from two-carbon linkage to ten-carbon linkage) in contrast to the sultone reactions that only yields three-carbon or four-carbon linkage; 2) accessibility to different cation and anion structures so that high dielectric constant can be synergized with other properties, such as better electrochemical stability, antibacterial properties, and lower viscosity; and 3) proper cation substitution which frustrates crystallization and enables the formation of zwitterionic liquids at ambient temperature. Though the dependency of dielectric constant on cation-anion linkage length or molecular dipole is obvious, obtaining high dielectric constant zwitterionic liquids at room temperature requires a proper molecular design that synergizes all the relevant molecular parameters, including ion substitution, cation, and anion chemical composition and linkage length. The disclosed synthesis demonstrates superior flexibility to tailor all the above-mentioned molecular parameters and yields zwitterionic liquids with the highest dielectric constant (˜400) among all the organic materials. The most effective way to tune the zwitterionic liquid dielectric constant is increasing cation-anion linkage length, which is realizable with the disclosed synthesis.

### Example 1

The general synthesis scheme is shown in Scheme I.

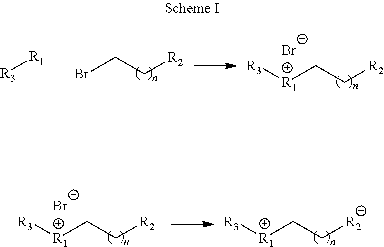

The exemplary and unlimiting synthesis involves two steps. The first step is a quaternization reaction with the nucleophiles R₃-R₂ and a brominated reagent Br—CH₂—(CH₂)ₙ—R₂, followed by an ion-exchange step that gives the zwitterion molecules. The nucleophile R₃-R₁, where R₁ could be amine, imidazole, pyrrolidine, piperidine, phosphine, etc., yields ammonium, imidazolium, pyrrolidinium, piperidinium, and phosphonium cation, and the R₃ is the substitution groups which can be methoxyethyl, (2-methoxyethoxy)ethyl, or [2-(2-methoxyethoxy)ethoxy]ethyl, alkyl, siloxanes (—(Si—O)₁)—, poly(ethylene oxides) (—(CH₂CH₂O)ₙ—, crown ether, etc. We will show the importance of incorporating the ethylene-oxide (EO) based substitution in the discussion part. For the brominated reagent Br—CH₂—(CH₂)—R₂, n could be varied between 1 to 10, determining the linkage length between the cation and anion, and R₂ could be the ester group or the ester group, the trifluoromethylsulfonylimide group to yield either carboxyl or sulfonylimide anion. Br—CH₂—(CH₂)ₙ—R₂ and Br—CH₂(CH₂)ₙ—COOCH₂CH₃ are commercially available, and the alkyl linkage could be replaced with the more flexible siloxanes (—(Si—O)ₙ)—, poly(ethylene oxides) (—(CH₂CH₂O)ₙ— linkage with a few more reaction steps.

The zwitterion structures that can be formed by the disclosed synthesis are shown in Table 1.

TABLE 1
Zwitterion structures accessible with the disclosed synthesis.

| | | Cation | |
|---|---|---|---|
| Cation^{a,b,e} | Anion^{a,e} | Substitute^{e} | Linkage^{e} |
| 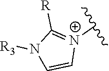 | 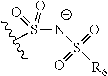 | 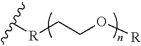 | 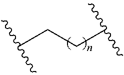 |
| 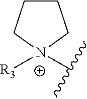 | 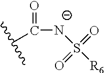 | 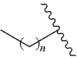 | 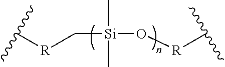 |
| 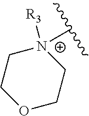 | 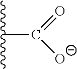 [SMILES: *C(=O)[O-]] | 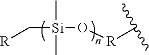 | 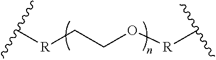 |
| 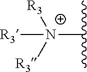 | 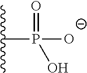 [SMILES: *P(=O)([O-])O] | 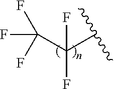 | 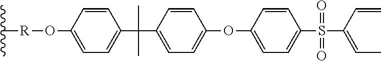 |
| 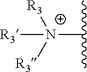 | 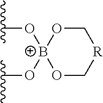 | 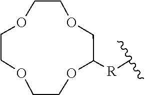 | 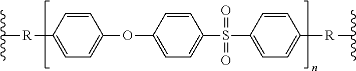 |
| 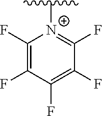 [SMILES: *[n+]1c(F)c(F)c(F)c(F)c1F] | 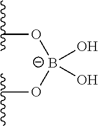 [SMILES: *O[B-](O)(O)O*] | 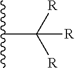 | 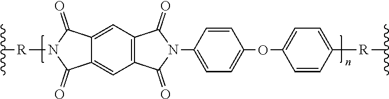 |
| | 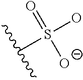 | | 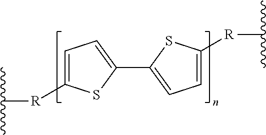 |

^{a}n could be between 2-100
^{b}R is the functional group which could be —H, alkyl, etc. depending on the reagent.
^{c}O atom could be substituted with CH₂
^{d}R₃, R₃′, and R₃″ could be the same or different.
^{e}R depends on the reagents used for functionalizing, which could be —H, alkyl, ethylene oxide, carbonyl, etc.
^{f}R₅ could be CF₃, —(CF₂)ₙCF₃, substituted phenyl, etc.
^{g}4-crown-12 ether as an example and could be any crown ether

In yet further aspects, cations can be selected from

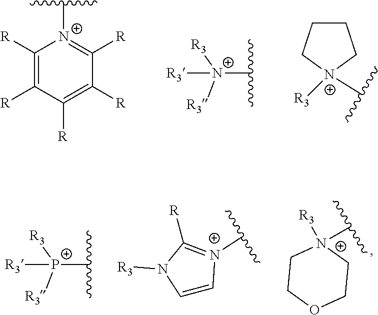

where O can be substituted with CH₂.

In still further aspects, cation substitutes can be

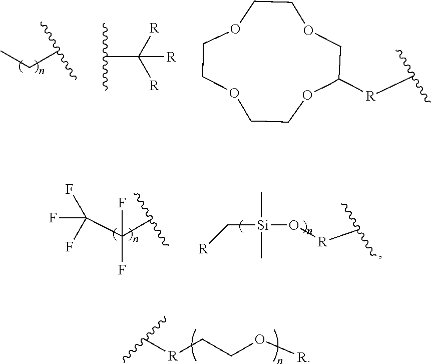

In still further aspects, linkages can be selected from

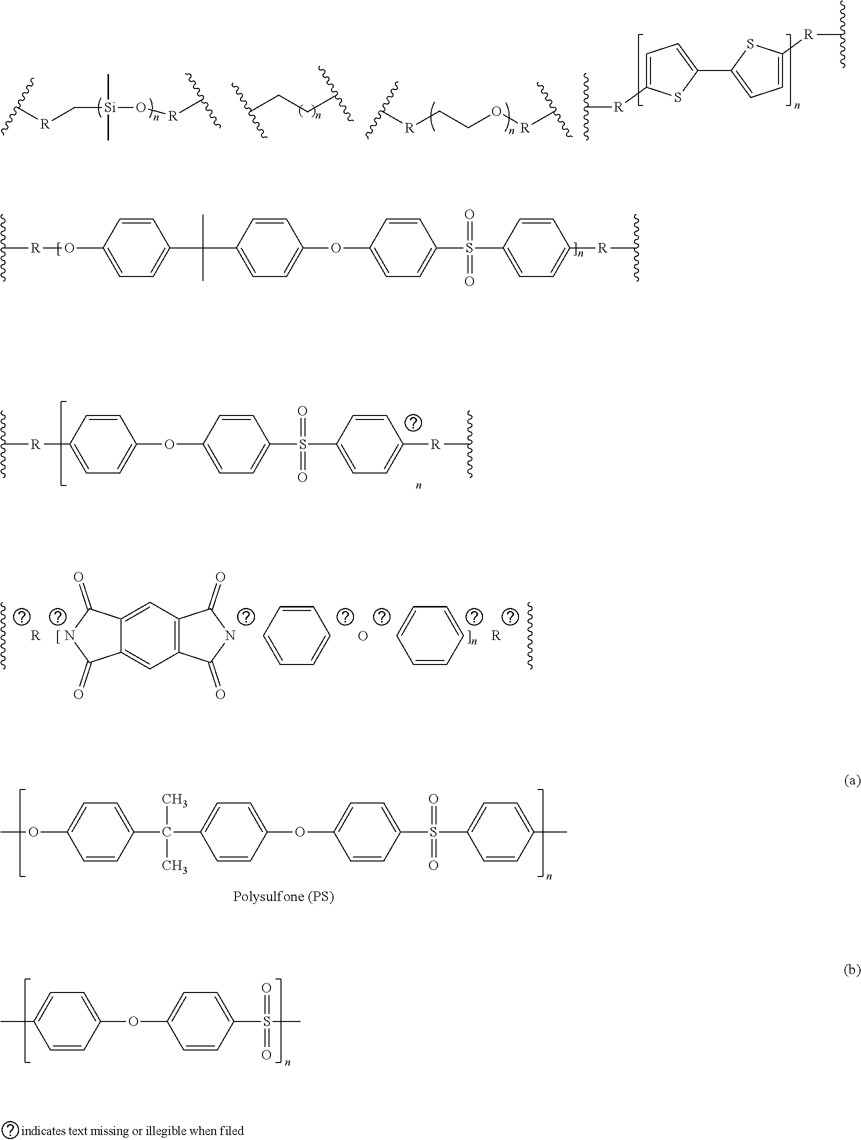

Polyaryletheresulfone (PAES)

In still further aspects, anions can be selected from

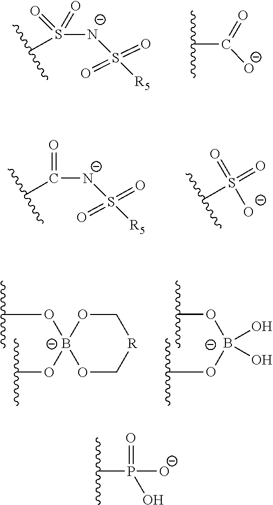

The disclosed synthesis yields high purity and a decent yield of zwitterions due to increased reaction selectivity and efficiency. For example, the yield is improved from 8% to 40% compared with the literature approach. Additionally, the advantages of this disclosed synthesis compared with the reported one are 1) use of triethylamine (TEA) reduces the amount of R₃-R₁ needed (only 1 equivalent amount to Br—(CH₂)ₙ—R₂ rather than 3) and hence lowers the cost significantly. R₃-R₁ is usually the most expensive chemical (e.g., 500 $ (25 g) for the Br—(CH₂—CH₂—O)₂—CH₃ vs. 60 $ (500 ml) for TEA). 2) the easy removal of TEA enables high product purity, especially for the zwitterions with long, complicated cation substitutes, because R₃-R₁ is more challenging to remove with increasing molecular weight.

### Example 2

### Synthesis of Imidazolium-Sulfonylimide Zwitterion

The reaction scheme is shown in Scheme II.

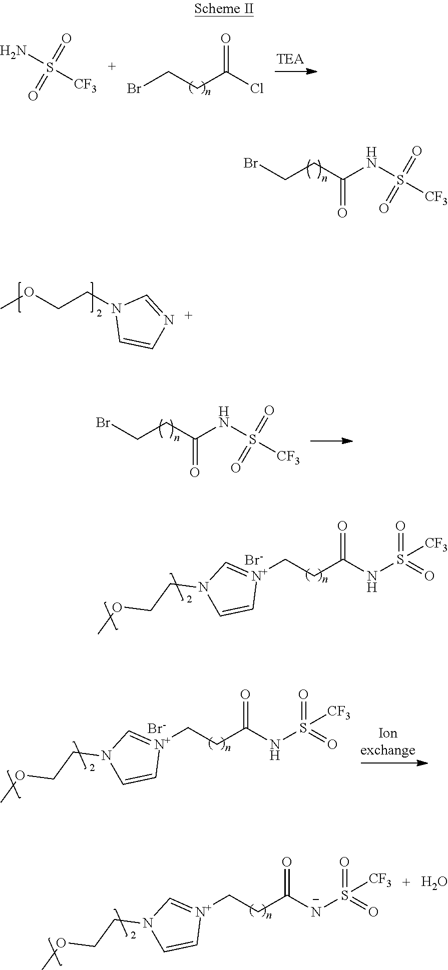

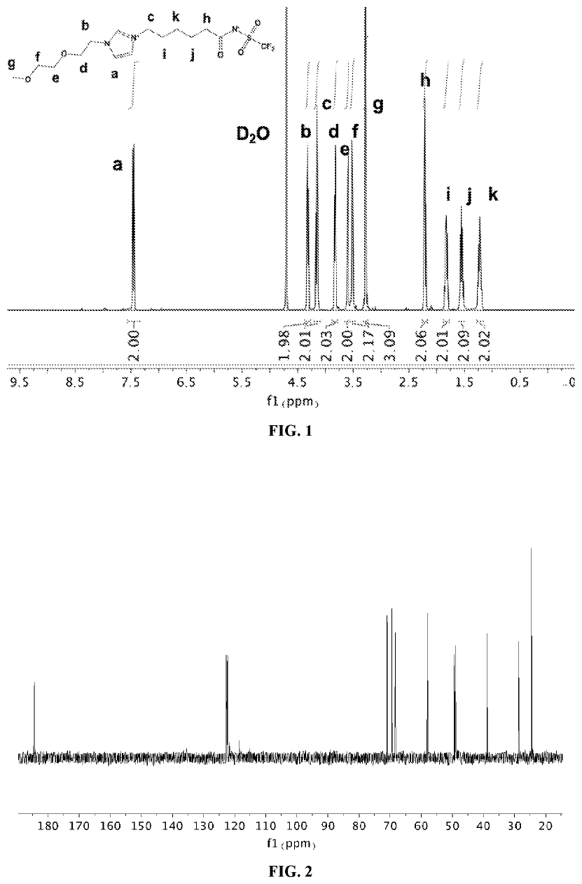

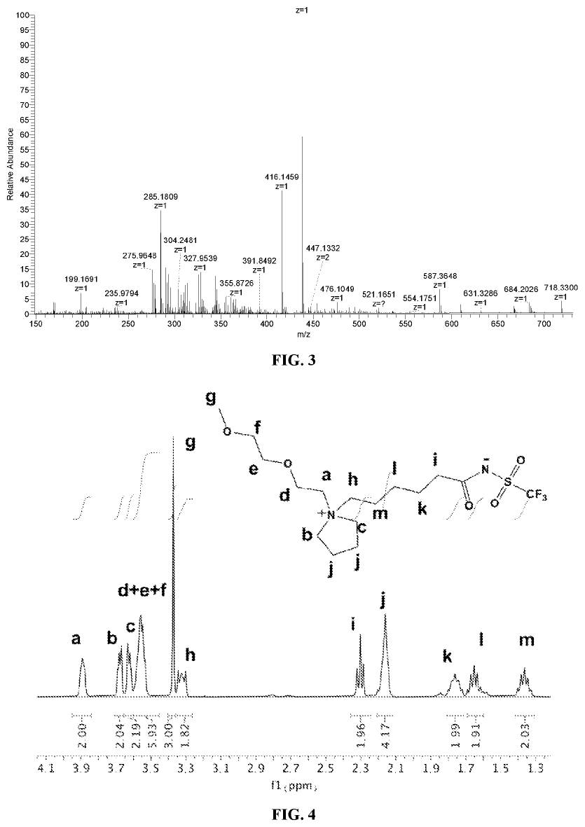

Synthesis procedures are given using 6-bromo-n-hexanoylchloride (n=4) and can be easily adapted with other reagents with different n. First, trifluromethanesulfonamide (2.1 g) and 6-bromo-n-hexanoylchloride (3.0 g) were reacted in the presence of triethylamine (2.9 g) at 0° C. for 24 hours. Then, the solution was condensed and re-dissolved in ethyl acetate and washed with saturated NaCl water five times. The organic layer was further concentrated with a rotatory evaporator followed by vacuum drying to yield the white powder (3.4 g, 67%). Next, the product from the first reaction was reacted with 1-(2-(2-Methoxyethoxy)ethyl)-1H-imidazole in anhydrous THF under argon at 45° C. for five days to obtain a colorless viscous liquid. The viscous solution was washed with diethyl ether five times and dried under a vacuum at 40° C. Then, the thoroughly dried liquid obtained from the second step was dissolved in deionized water and passed through an anion-exchange column to yield the neutral zwitterion. The water was removed by freeze-drying for two days, followed by vacuum drying at 80° C. for 36 hours. The molecular structure was confirmed with ¹H NMR, ¹³C NMR, and TOF-mass spectrometry shown in FIGS. 1-3, confirming the molecular structure and product purity.

### Example 3

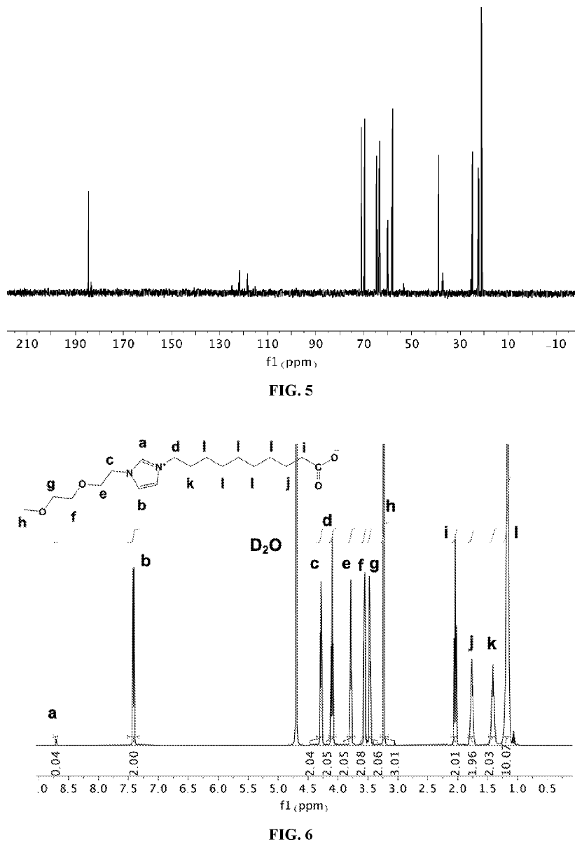

The second example is given for the synthesis of pyrrolidium-sulfonylimide zwitterion. The imidazole used in the first example (Scheme III) is replaced with pyrrolidine with the same experimental protocols. The molecular structure of the final product is confirmed with ¹H NMR, ¹³C NMR shown in FIGS. 4-5.

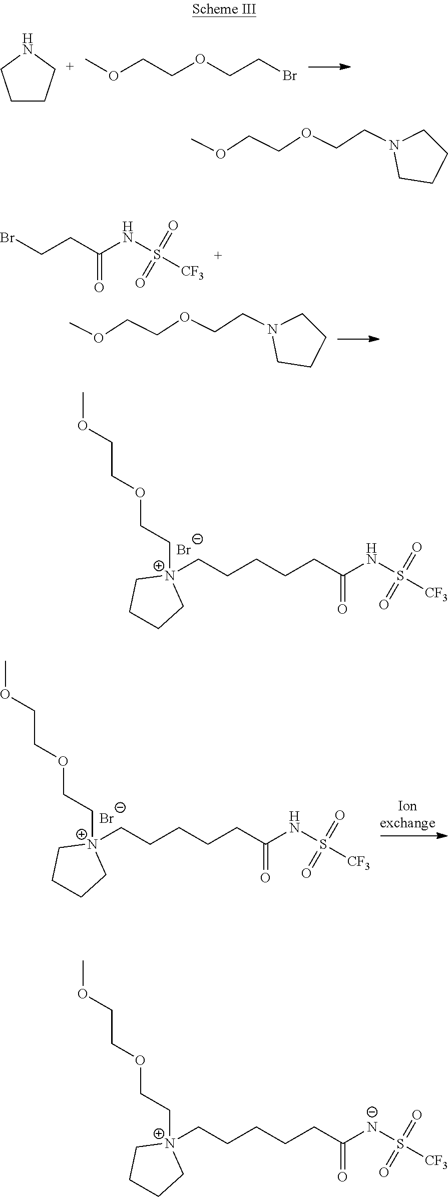

### Example 4

The third example is given for the synthesis of imidazolium-carboxyl zwitterion. The synthesis scheme is detailed in Scheme III. The brominated reagent is ethyl 11-bromoundecanoate (n=8). ¹H NMR confirms the molecular structure (FIG. 6).

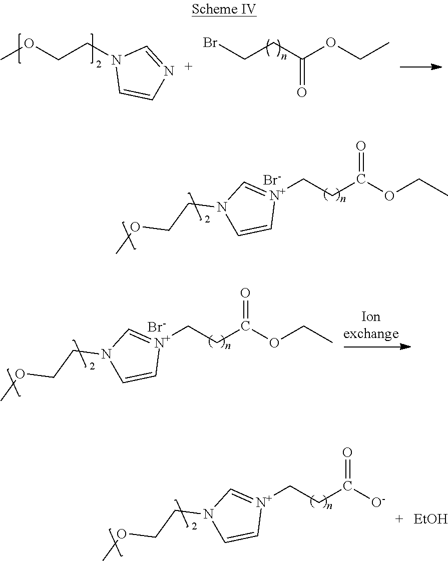

### Results and Discussion

FIG. 7 demonstrates the temperature dependence of the dielectric constant of zwitterionic liquids. The zwitterions are named to indicate the cation substituents, the cation, the number of carbons between cation and anion, and anion. For example, OE2Im6N (FIG. 1) is the zwitterion with (2-methoxyethoxy)ethyl (OE2) cation substituted imidazolium (Im) with six-carbon linkage (6) between the imidazolium (Im) and sulfonylimide anion (N). Py represents the pyrrolidium cation (FIG. 4), N represents ammonium cation (OE6N3S), and S represents the sulfonate anion. Data for sulfonate zwitterion is from previous publications and is included for comparison.

Zwitterionic liquids can achieve a high dielectric constant (>300) at ambient temperature, as demonstrated by OE2Im6N and OE2Py6N with the long cation-anion linkage (6-carbon). The higher dielectric constant of OE2Im6N than OE2Im4S and OE2Im3S indicates that the dielectric constant is tunable through linkage length due to different molecular dipoles. However, longer linkage length does not guarantee a higher dielectric constant, as the cation substitution and ion chemical compositions also impact the dielectric constant of zwitterionic liquids. For example, zwitterions with (2-methoxyethoxy)ethyl cation substitution (OE2Im4S and OE2Im3S) show more than double the dielectric constant than their alkyl cation substitution counterpart (OctIm4S and OctIm3S) because (2-methoxyethoxy)ethyl substitution renders more homogenous zwitterionic liquid structures compared with the alkyl counterpart.

Suppressing crystallization can be needed for high dielectric constant zwitterionic liquids, as demonstrated by BuIm4S, BuIm3S, and OE6N4S which are solids at ambient temperature. The high dielectric constant of BuIm3S and BuIm4S (Bu represents butyl cation substitute) is only accessible at elevated temperatures above their Tₘ (>150° C., open circles, and open diamond). Crystallization decreases the dielectric constant significantly, as illustrated by semi-crystalline OE6N4S (open triangle) and supercooled liquid OE63S (filled triangle). The former crystallizes immediately during cooling and only shows half of the dielectric constant than that of OE6N3S at 30° C. despite its longer linkage length (4-carbon vs. 3-carbon).

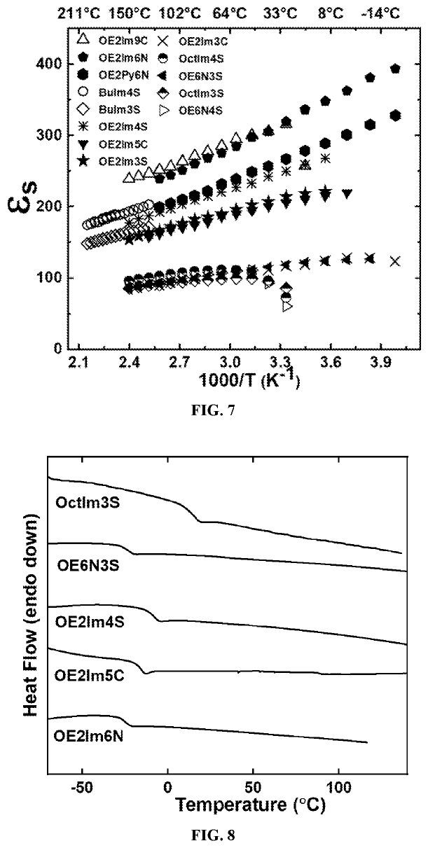

Differential Scanning Calorimetry (DSC) data further confirms the formation of zwitterionic liquids at room temperature and implicates the benefits of proper cation substitution on zwitterion properties. As shown in FIG. 8, zwitterions with ethylene-oxide cation substitution (OE2 or OE6) show lower Tg regardless of the anion structures than the alkyl counterpart and frustrated crystallization. The only known example for alkyl-substituted zwitterion that does not crystallize rapidly is OctIm3S. However, the alkyl substitutes increase the Tg and considerably lower the dielectric constant.

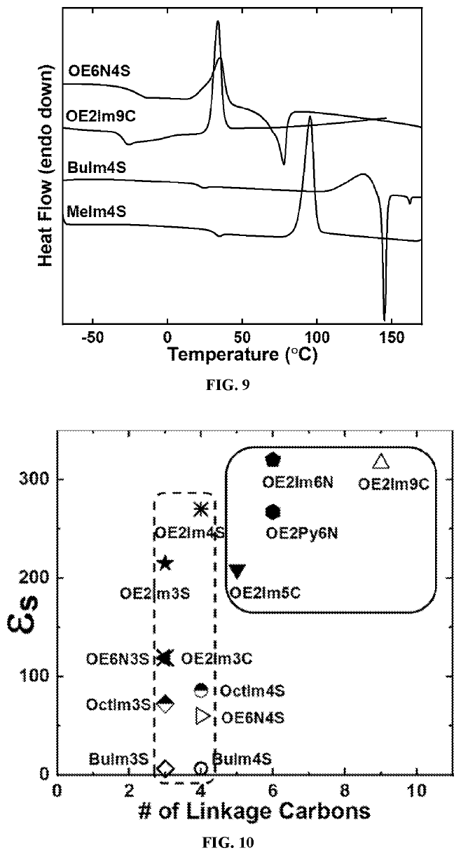

Most zwitterions reported crystallizing rapidly at ambient temperatures. A few examples are given in FIG. 9, which shows the rapid crystallization during the DSC measurements (less than 3 hours). In addition to proper cation substitutes discussed above, anion structure and linkage are also important. For example, zwitterions with more charge localized anion structures such as sulfonate or carboxyl crystallize easier than the more charge delocalized sulfonylimide anion. Very long linkage length (e.g., OE2Im9C) facilitates crystallization. Yet, the melting temperature is significantly lowered due to the EO-based cation functionalization (35° C. for OE2Im9C vs. 140° C. for EtIm9C), reiterating the importance of proper cation functionalization. The molecular design that enables the formation of zwitterionic liquids at ambient temperatures likely requires: 1) proper cation substitutes (e.g., (—(CH₂CH₂O)ₙ—), 2) charge delocalized ion structures (e.g., imidazolium, sulfonylimide), and 3) moderate linkage length (e.g., 5-10 carbon spacer).

FIG. 10 demonstrates the potency of the disclosed synthesis to achieve high dielectric constant zwitterionic liquids by comparing the dielectric constant of zwitterions at 30° C. Zwitterions synthesized via the disclosed synthesis routes are indicated in solid square, with high dielectric constant and versatile cation/anion combinations. In contrast, the common ring-opening sultone chemistry only yields zwitterions with limited linkage length (three-carbon or four-carbon linkage, indicated with dashed square), and only the cation structure can be varied. FIG. 10 also highlights the importance of proper cation substitutions to suppress crystallization, as the zwitterions that crystallize rapidly show a low dielectric constant (see open symbols for OE6N4S, BuIm3S, and BuIm4S). Linkage length is the most effective parameter that impacts the dielectric constant. Compared with other synthesis methods, the disclosed synthesis results in zwitterion with tunable longer linkage length, a wide range of cation/anion combinations, and desirable ion functionalities with reduced cost using cheaper reagents. Hence, the disclosed synthesis method is the most potent method to synthesize zwitterionic liquids with a high dielectric constant compared with other methods.

Overall, the disclosed synthesis method yields high dielectric constant zwitterionic liquids. High reaction selectivity and efficiency ensure high purity and good yield for the reaction products. The disclosed synthesis method could result in a broad range of zwitterions, as exampled in Table 1. The tunability of linkage length, the flexibility of incorporating different cation substitutions, and the versatility of cation/anion chemical compositions provided with the disclosed synthesis yield not only high dielectric constant zwitterionic liquids but also enable other functionalities that could be synergized for a wide range of applications.

### Example 5

The following structures were synthesized by the disclosed herein methods

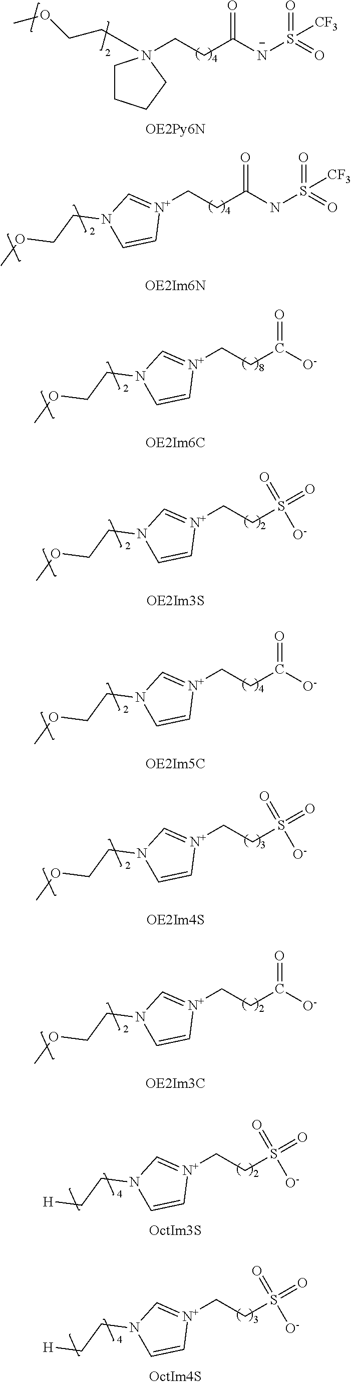

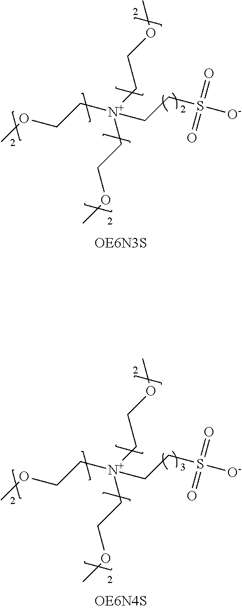

### Example 5

The following structures can be synthesized by the disclosed herein methods

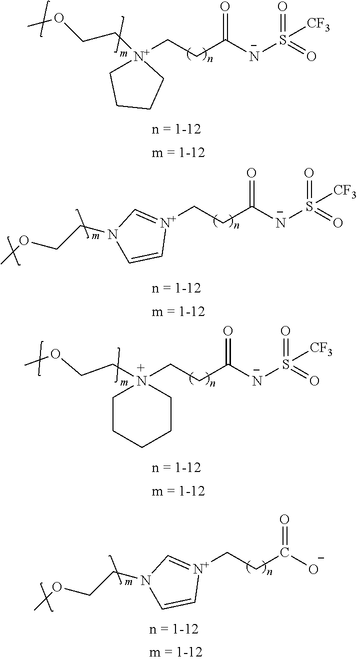

### CONCLUSION

A versatile, effective, and highly selective synthesis method for high dielectric constant zwitterionic liquids with tailorable chemical composition was developed. Specifically, the disclosed synthesis method provides excellent flexibility to tune the length of the cation-anion linkage, thus resulting in a considerably larger dielectric constant due to the longer cation-anion linkage, which is not accessible with commonly used ring-opening chemistries. A wide range of cation/anion chemical compositions are accessible to synergize other functionalities with a high dielectric constant. Proper cation substitution helps suppress crystallization and increase molecular mobility. The disclosed synthesis method provides a library of high dielectric constant zwitterionic liquids that can be used in various applications.

The claims are not intended to include, and should not be interpreted to include, means-plus- or step-plus-function limitations unless such a limitation is explicitly recited in a given claim using the phrase(s) “means for” or “step for,” respectively.

In view of the described processes and compositions, hereinbelow are described certain more particularly described aspects of the inventions. These particularly recited aspects should not, however, be interpreted to have any limiting effect on any different claims containing different or more general teachings described herein or that the “particular” aspects are somehow limited in some way other than the inherent meanings of the language and formulas literally used therein.

### REFERENCES

¹ Q. Chen, Y. Shen, S. Zhang, and Q. M. Zhang, Annu. Rev. Mater. Res. 45 (2015) 433.
² Q. Zhang, H. Li, M. Poh, F. Xia, Z.-Y. Cheng, H. Xu, and C. Huang, Nature 419 (2002) 284.
³ A. Javey, H. Kim, M. Brink, Q. Wang, A. Ural, J. Guo, P. McIntyre, P. McEuen, M. Lundstrom, and H. Dai, Nat. Mater. 1 (2002) 241.
⁴ D. Zhou, D. Shanmukaraj, A. Tkacheva, M. Armand, and G. Wang, Chem 5 (2019) 2326.
⁵ Q. Zhao, S. Stalin, C.-Z. Zhao, and L. A. Archer, Nat. Rev. Mater. 5 (2020) 229.
⁶ L. Zhu, J. Phys. Chem. Lett. 5 (2014) 3677.
⁷ O. Kim, H. Kim, U. H. Choi, and M. J. Park, Nat. Commun. 7 (2016) 13576.
⁸ Y. Wang, X. Zhou, Q. Chen, B. Chu, and Q. Zhang, IEEE Trans. Dielectr. Electr. Insul. 17 (2010) 1036.
⁹ B. Boz, T. Dev, A. Salvadori, and J. L. Schaefer, J. Electrochem. Soc. 168 (2021) 090501.
¹⁰ V. Bocharova, and A. P. Sokolov, Macromolecules (2020)
¹¹ E. R. Logan, and J. R. Dahn, Trends in Chemistry 2 (2020) 354.
¹² C. Y Son, and Z.-G. Wang, J. Chem. Phys. 153 (2020) 100903.
¹³ B. Onyenucheya, J. Allen, K. Pierre, J. Zimheld, and K. Burke, in 2019 IEEE Pulsed Power & Plasma Science (PPPS) 2019), pp. 1.
¹⁴ L. J. Romasanta, M. A. Lopez-Manchado, and R. Verdejo, Prog. Polym. Sci. 51 (2015) 188.
¹⁵ A. O'Halloran, F. O'Malley, and P. McHugh, J. Appl. Phys. 104 (2008) 071101.
¹⁶ W Mei, A. J. Rothenberger, J. E. Bostwick, J. M. Rinehart, R. J. Hickey, and R. H. Colby, Phys. Rev. Lett. 127 (2021) 228001.
¹⁷ W Mei, A. Han, R. J. Hickey, and R. H. Colby, J. Chem. Phys. 155 (2021) 244505.
¹⁸ H. Ohno, M. Yoshizawa-Fujita, and Y. Kohno, Bull. Chem. Soc. Jpn. 92 (2019) 852.
¹⁹ H. Ohno, Electrochemical Aspects of Ionic Liquids 2005),
²⁰ M. Yoshizawa, A. Narita, and H. Ohno, Aust. J. Chem. 57 (2004) 139.
²¹ H. Ohno, M. Yoshizawa-Fujita, and Y. Kohno, Phys. Chem. Chem. Phys. 20 (2018) 10978.
²² M. Yoshizawa-Fujita, A. Narita, and H. Ohno, in Electrochemical Aspects of Ionic Liquids, edited by H. Ohno (John Wiley & Sons, Inc., 2011), pp. 301.
²³ H. Ohno, Bull. Chem. Soc. Jpn. 79 (2006) 1665.
²⁴ Z. Huang, S. Choudhury, H. Gong, Y. Cui, and Z. Bao, J. Am. Chem. Soc. 142 (2020) 21393.
²⁵ N. Nikfarjam, M. Ghomi, T. Agarwal, M. Hassanpour, E. Sharifi, D. Khorsandi, M. Ali Khan, F. Rossi, A. Rossetti, E. Nazarzadeh Zare, N. Rabiee, D. Afshar, M. Vosough, T. Kumar Maiti, V. Mattoli, E. Lichtfouse, F. R. Tay, and P. Makvandi, Adv. Funct. Mater. 31 (2021) 2104148.
²⁶ W Zhong, Z. Shi, S. H. Mahadevegowda, B. Liu, K. Zhang, C. H. Koh, L. Ruan, Y. Chen, M. S. Zeden, C. J. E. Pee, K. Marimuthu, P. P. De, O. T. Ng, Y Zhu, Y. R. Chi, P. T. Hammond, L. Yang, Y.-H. Gan, K. Pethe, E. P. Greenberg, A. Grundling, and M. B. Chan-Park, Proc. Natl. Acad. Sci. 117 (2020) 31376.
²⁷ M. Yoshizawa-Fujita, T. Tamura, Y Takeoka, and M. Rikukawa, Chem. Commun. 47 (2011) 2345.
²⁸ A. Narita, W. Shibayama, and H. Ohno, J. Mater. Chem. 16 (2006) 1475.
²⁹ J. P. Uittenbogaard, B. Zomer, P. Hoogerhout, and B. Metz, J. Biol. Chem. 286 (2011) 36198.

### ASPECTS

Aspect 1: A compound of Formula (I):

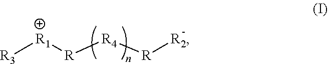

wherein
   R is null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl,
   R₁ is selected from substituted or unsubstituted ammonium, imidazolium, pyrrolidinium, piperidinium, boronium, and phosphonium cation,
   R₂ is selected from

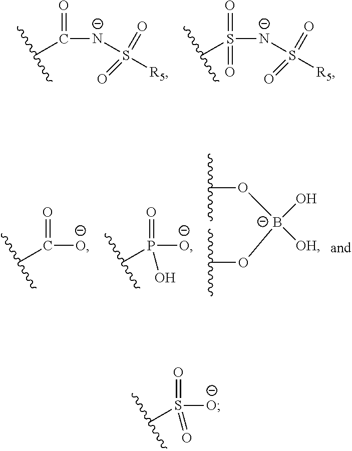

R₃ is selected from methoxyethyl, (2-methoxyethoxy)ethyl, or [2-(2-methoxyethoxy)ethoxy]ethyl, alkyl, a siloxane, a polyethylene oxide, and

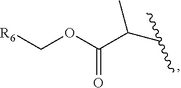

R₅ is selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, ether, and fluoroalkyl; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, trifluoromethyl, difluoromethylene, or phosphonyl,
R₆ is a substituted or unsubstituted crown ether, polyethylene(oxide) or siloxane,
R₄ is selected from —CH₂—, a siloxane, polysulfone, polyimide, polythiophene a polyethylene oxide, and —R—O—; and
n is an integer selected from 1 to 30,
wherein the compound of Formula (I) is zwitterionic and exhibits a dielectric constant higher than from about 100 to about 2000 at a temperature from about 20° C. to about 70° C.
Aspect 2: The compound of Aspect 1, wherein R₅ is —CF₃, —(CH₂CH₂)CF₃, or —(CF₂)₇CF₃.
Aspect 3: The compound of Aspect 1 or 2, wherein the substituted or unsubstituted crown ether can be selected from

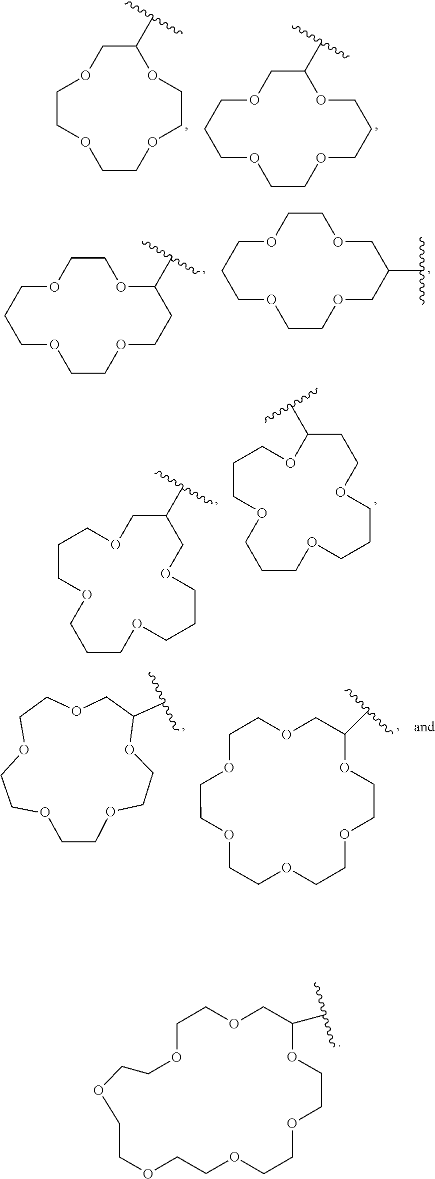

Aspect 4: The compound of any one of Aspects 1-3, wherein the compound of Formula (I) is a liquid at a temperature from about 20° C. to about 70° C. for at least about 1 month.
Aspect 5: The compound of any one of Aspects 1-4, wherein the compound of Formula (I) comprises one or more of

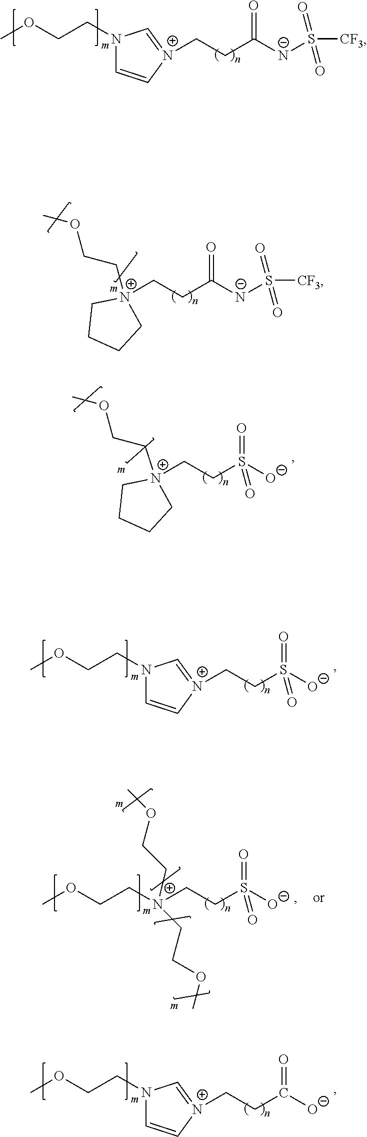

wherein m is from 1 to 6 and n is from 2 to 8.
Aspect 6: The compound of any one of Aspects 1-5, wherein the compound of Formula (I) comprises

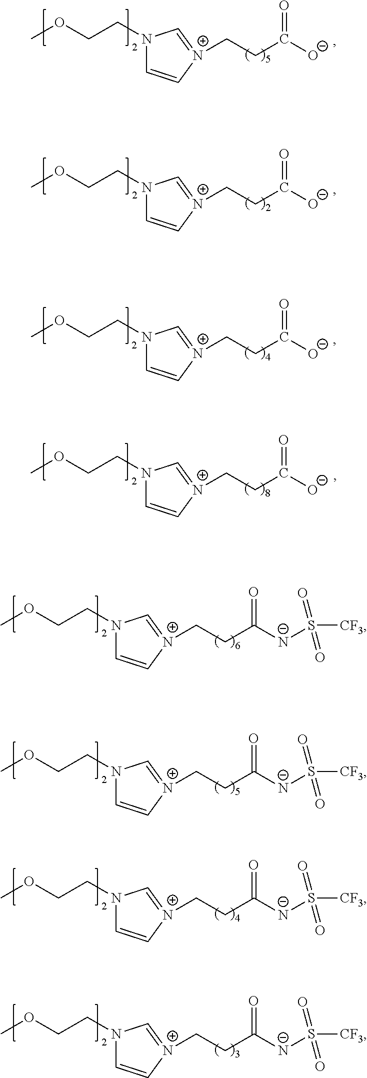

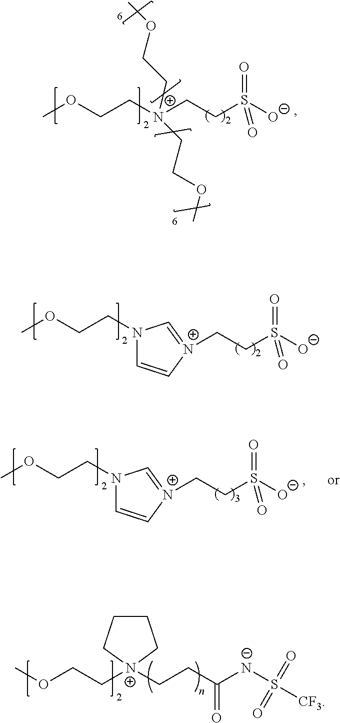

Aspect 7: The compound of Aspect 5, wherein the compound of Formula (I) is substantially free of crystallization for about 1 to about 12 months at a temperature from about 20° C. to about 70° C.
Aspect 8: A device comprising the compound of any one of Aspects 1-7.
Aspect 9: The device of Aspect 8, wherein the device is a battery, an electronic device, a soft robotics device, a dielectric elastomer, a capacitor, or any combination thereof
Aspect 10: A method of making a compound of Formula (I)

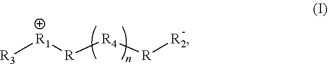

comprising:
a) reacting a compound of Formula (II)

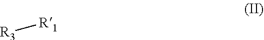

with a reagent of Formula (III)

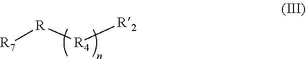

to form an intermediate compound of Formula (IV)

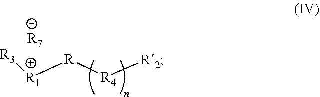

b) performing an ion-exchange of the compound (IV) to form the compound of Formula (I), wherein
   R is null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl,
   R′₁ is selected from a substituted or unsubstituted amino group, imidazole group, pyrrolidine group, piperidine group, aminoborane group, and phosphine group,
   R₁ is selected from substituted or unsubstituted ammonium, imidazolium, pyrrolidinium, piperidinium, boronium, and phosphonium cation,
R′₂ is selected from

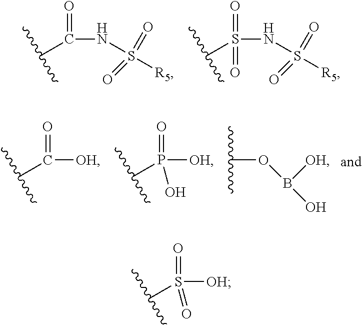

R₂ selected from

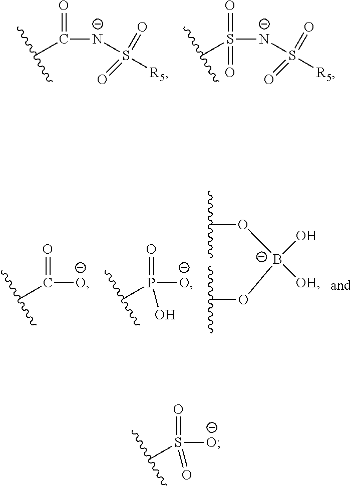

R₃ is selected from methoxyethyl, (2-methoxyethoxy)ethyl, or [2-(2-methoxyethoxy)ethoxy]ethyl, alkyl, a siloxane, a poly(ethylene oxide), and

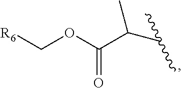

R₅ is selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, ether, and fluoroalkyl; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, trifluoromethyl, difluoromethylene, and phosphonyl,
R₆ is a substituted or unsubstituted crown ether, polyethylene(oxide) or siloxane,
R₄ is selected from —CH₂—, a siloxane, polysulfone, polyimide, polythiophene, a polyethylene oxide, and —R—O—;
   n is an integer selected from 1 to 30, and
   R₇ is a leaving group comprising a halogen;
   wherein the compound of Formula (I) is zwitterionic and exhibits a dielectric constant higher than from about 100 to about 2000 at a temperature from about 20° C. to about 70° C.

Aspect 11: The method of Aspect 10, wherein R₅ is —CF₃, —(CH₂CH₂)CF₃, or —(CF₂)₇CF₃.

Aspect 12: The method of Aspect 10 or 11, wherein the reagent of Formula (III) is formed by reacting compound of Formula (V)

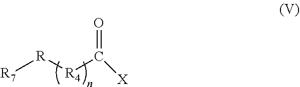

with a compound of Formula (VI)

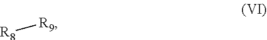

in the presence of a base,
   wherein X is a halogen, and
   R₈ is selected from H, C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; wherein R⁸ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl, and
   R₉ is independently selected from —NH₂, —OH, and —COO⁻.
Aspect 13: The method of Aspect 12, the base comprises triethylamine.
Aspect 14: The method of any one of Aspects 10-13, wherein the substituted or unsubstituted crown ether can be selected from

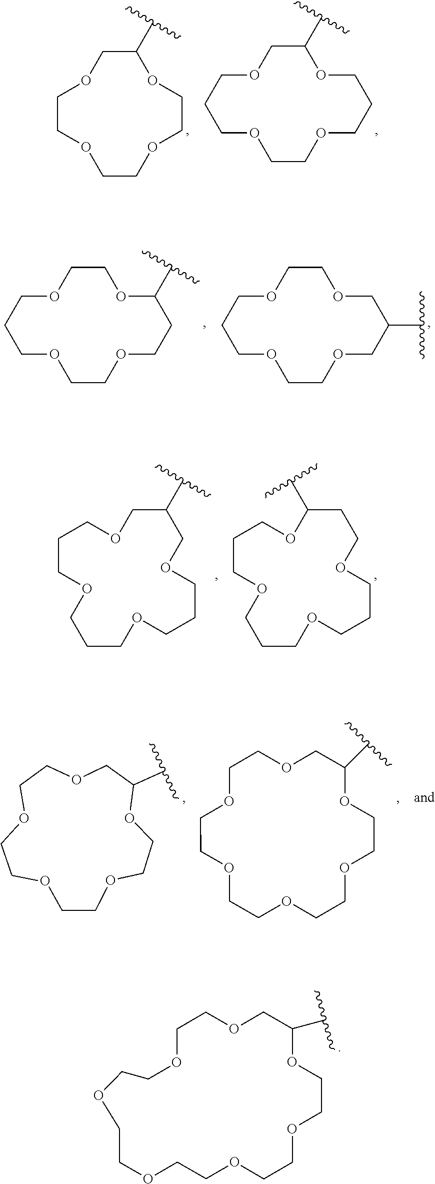

Aspect 15: The method of any one of Aspects 10-14, wherein the compound of Formula (I) is a temperature from about 20° C. to about 70° C. for at least about 1 month.
Aspect 16: The method of any one of Aspects 10-15, wherein the compound of Formula (I) comprises one or more of

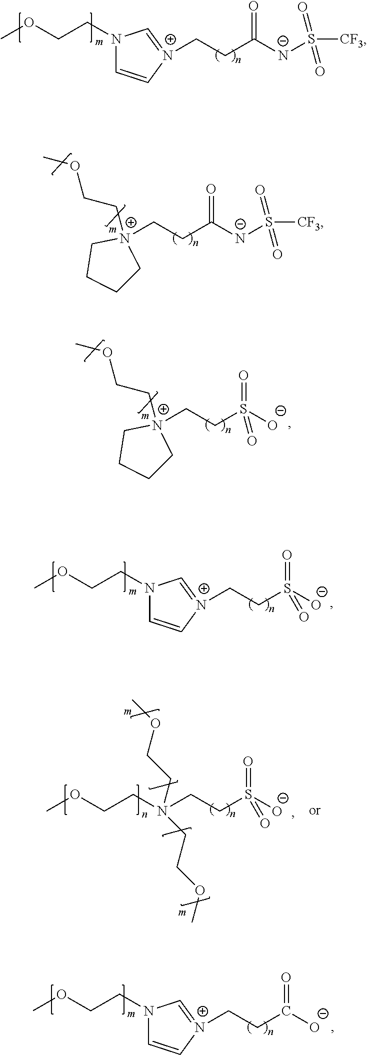

and
   wherein in is from 1 to 6 and n is from 2 to 8.
Aspect 17: The method of any one of Aspects 10-16, wherein the compound of Formula (I) comprises

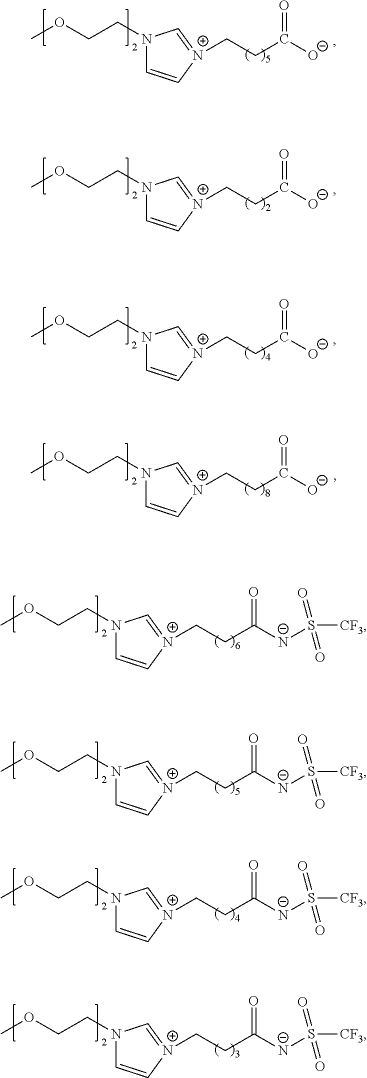

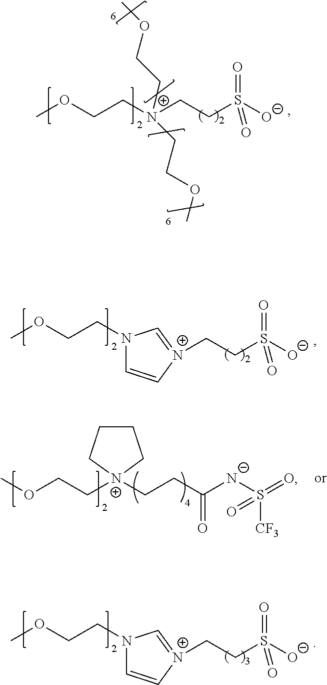

Aspect 18: The method of Aspect 17, wherein the compound of Formula (I) is free of crystallization for about 1 to about 12 months at a temperature from about 20° C. to about 70° C.
Aspect 19: The method of any one of Aspects 10-18, wherein the compound of Formula (I) of Formula (I) is formed with a yield from about 10% to 100%.
Aspect 20: The method of Aspect 19, wherein the yield is greater than about 40%.

1. A compound of Formula (I): 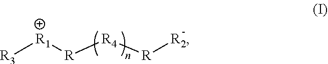
wherein
R is null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl,
R₁ is selected from a substituted or unsubstituted ammonium, imidazolium, pyrrolidinium, piperidinium, boronium, and phosphonium cation,
R₂ is selected from 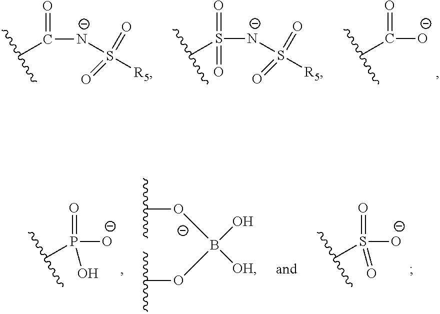
R₃ is selected from methoxyethyl, (2-methoxyethoxy)ethyl, or [2-(2-methoxyethoxy)ethoxy]ethyl, alkyl, a siloxane, a polyethylene oxide, and 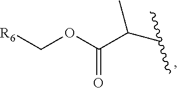
R₅ is selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, ether, and fluoroalkyl; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, trifluoromethyl, difluoromethylene, and phosphonyl,
R₆ is a substituted or unsubstituted crown ether, polyethylene(oxide) or siloxane;
R₄ is selected from —CH₂— a siloxane, polysulfone, polyimide, polythiophene, a polyethylene oxide, and —R—O—; and
n is an integer selected from 1 to 30,
wherein the compound of Formula (I) is zwitterionic and exhibits a dielectric constant higher than from about 100 to about 2000 at a temperature from about 20° C. to about 70° C.

2. The compound of claim 1, wherein R₅ is —CF₃, —(CH₂CH₂)CF₃, or —(CF₂)₇CF₃.

3. The compound of claim 1, wherein the substituted or unsubstituted crown ether can be selected from

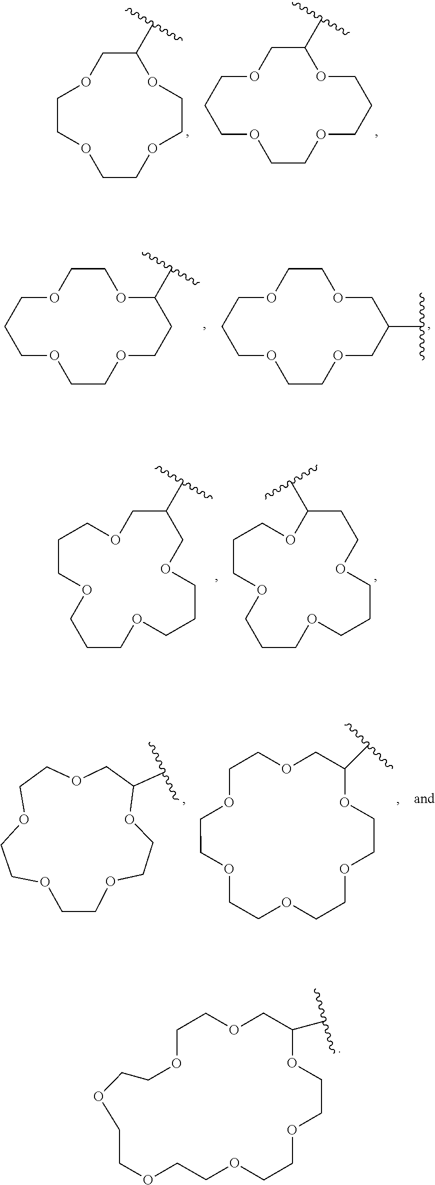

4. The compound of claim 1, wherein the compound of Formula (I) is a liquid at a temperature from about 20° C. to about 70° C. for at least about 1 month.

5. The compound of claim 1, wherein the compound of Formula (I) comprises one or more of 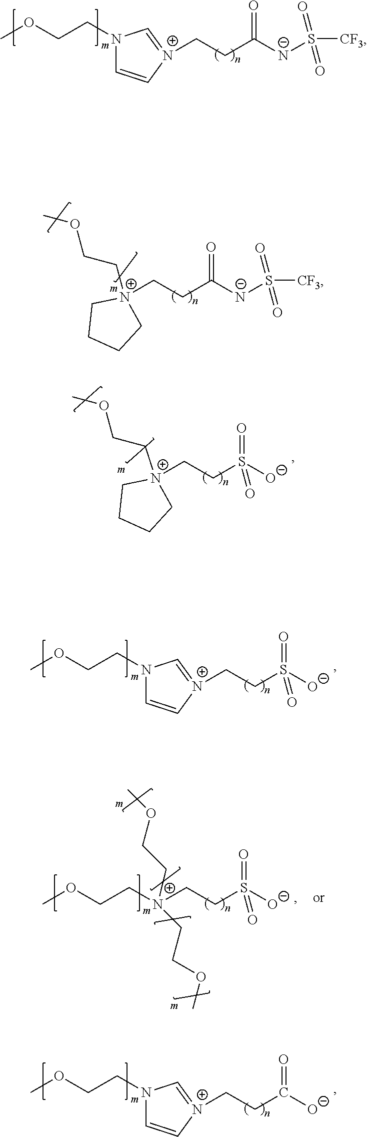
wherein m is from 1 to 6 and n is from 2 to 8.

6. The compound of claim 1, wherein the compound of Formula (I) comprises

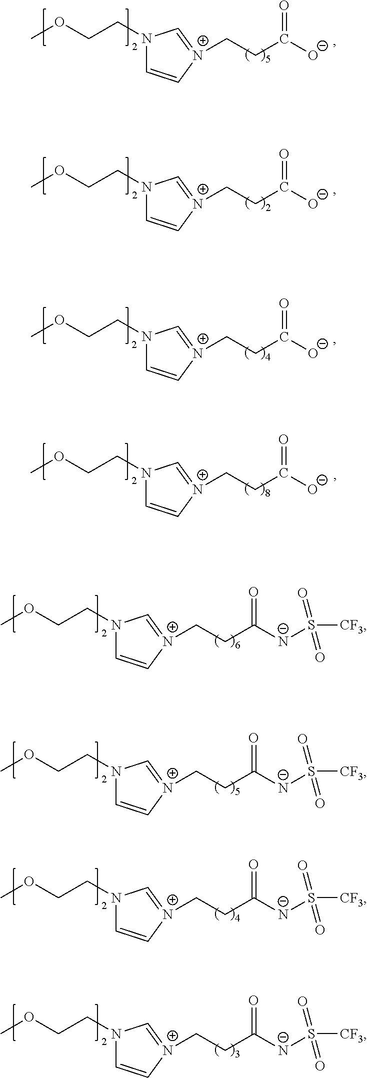

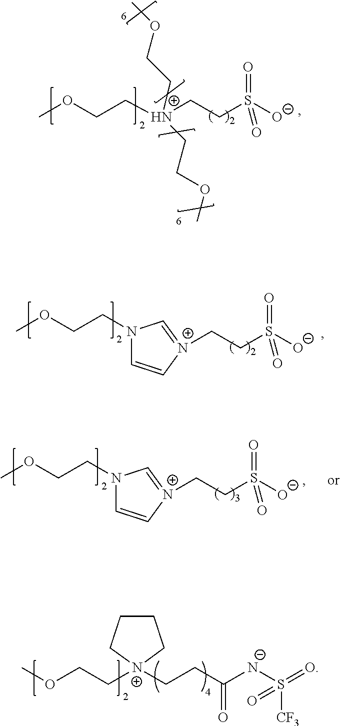

7. The compound of claim 5, wherein the compound of Formula (I) is substantially free of crystallization for about 1 to about 12 months.

8. A device comprising the compound of claim 1.

9. The device of claim 8, wherein the device is a battery, an electronic device, a soft robotics device, a dielectric elastomer, a capacitor, or any combination thereof.

10. A method of making a compound of Formula (I) 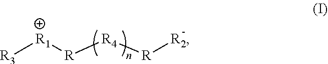
comprising:
a) reacting a compound of Formula (II) 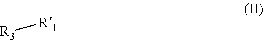
with
a reagent of Formula (III) 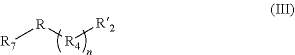
to form an intermediate compound of Formula (IV) 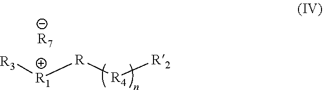
b) performing an ion-exchange of the compound of Formula (IV) to form the compound of Formula (I), wherein
R is null or selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl,
R′₁ is selected from a substituted or unsubstituted amino group, imidazole group, pyrrolidine group, piperidine group, aminoborane group, organoborane group, and phosphine group,
R₁ is selected from substituted or unsubstituted ammonium, imidazolium, pyrrolidinium, piperidinium, boronium, and phosphonium cation,
R′₂ is selected from 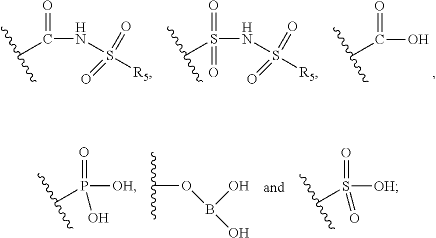
R₂ is selected from 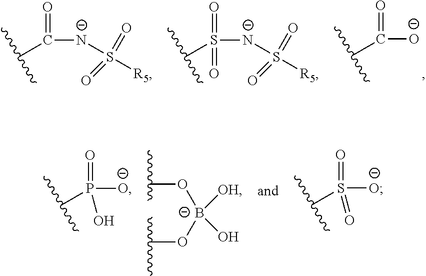
R₃ is selected from methoxyethyl, (2-methoxyethoxy)ethyl, or [2-(2-methoxyethoxy)ethoxy]ethyl, alkyl, a siloxane, a polyethylene oxide, and 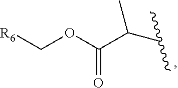
R₅ is selected from C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, C₆-C₁₄ aryloxy; ester, ether, and fluoroalkyl; wherein R₅ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, trifluoromethyl, difluoromethylene and phosphonyl,
R₆ is a substituted or unsubstituted crown ether, polyethylene(oxide) or siloxane;
R₄ is selected from —CH₂— a siloxane, polysulfone, polyimide, polythiophene, a polyethylene oxide, or —R—O—;
n is an integer selected from from 1 to 30, and
R₇ is a leaving group comprising a halogen;
wherein the compound of Formula (I) is zwitterionic and exhibits a dielectric constant higher from about 100 to about 2000 at a temperature from about 20° C. to about 70° C.

11. The method of claim 10, wherein R₅ is —CF₃, —(CH₂CH₂)CF₃, or —(CF₂)₇CF₃.

12. The method of claim 10, wherein the reagent of Formula (III) is formed by reacting a compound of Formula (V) 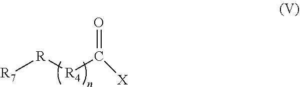
with a compound of Formula (VI) 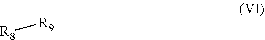
in the presence of a base,
wherein X is a halogen;
R₈ is selected from H, C₁₋₂₀ alkyl, C₂₋₂₀ alkenyl, C₁-C₂₀ alkoxy, C₂₋₂₀ alkynyl, C₁₋₂₀ heteroalkyl, C₂₋₂₀ heteroalkenyl, C₂₋₂₀ heteroalkynyl, C₆-C₁₄ aryl, C₁-C₁₃ heteroaryl, and C₆-C₁₄ aryloxy; wherein R⁸ is optionally substituted with one or more groups selected from C₁-C₂₀ alkyl, C₁-C₂₀ alkoxy, C₂-C₂₀ alkenyl, C₂-C₂₀ alkynyl, C₆-C₁₄ aryl, C₁-C₁₃ heteroaryl, amino, carbonyl, ester, ether, halide, carboxyl, hydroxy, nitro, cyano, silyl, sulfo-oxo, sulfonyl, sulfone, sulfoxide, thiol, and phosphonyl; and
R₉ is independently selected from —NH₂, —OH, and —COO⁻.

13. The method of claim 12, wherein the base comprises triethylamine.

14. The method of claim 10, wherein the substituted or unsubstituted crown ether can be selected from

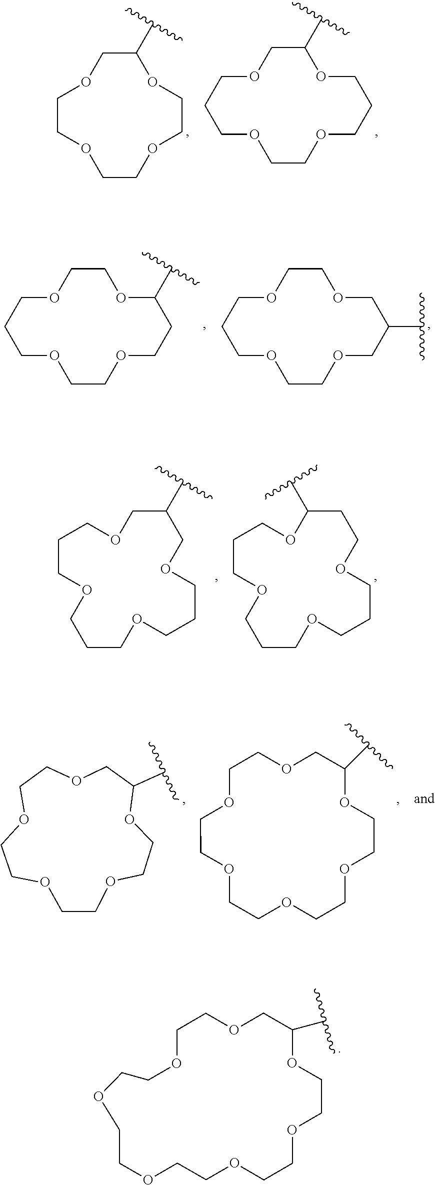

15. The method of claim 10, wherein the compound of Formula (I) is a liquid a temperature from about 20° C. to about 70° C. for at least about 1 month.

16. The method of claim 10, wherein the compound of Formula (I) comprises one or more of 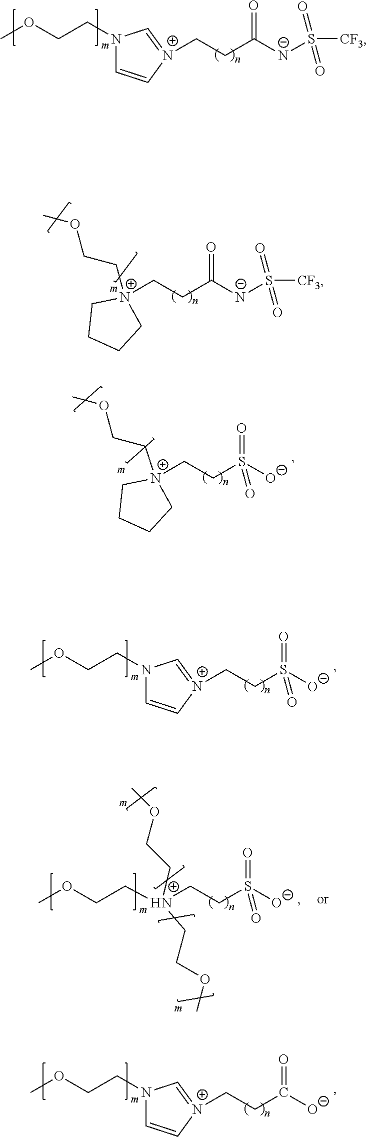
wherein m is from 1 to 6 and n is from 2 to 8.

17. The method of claim 10, wherein the compound of Formula (I) comprises

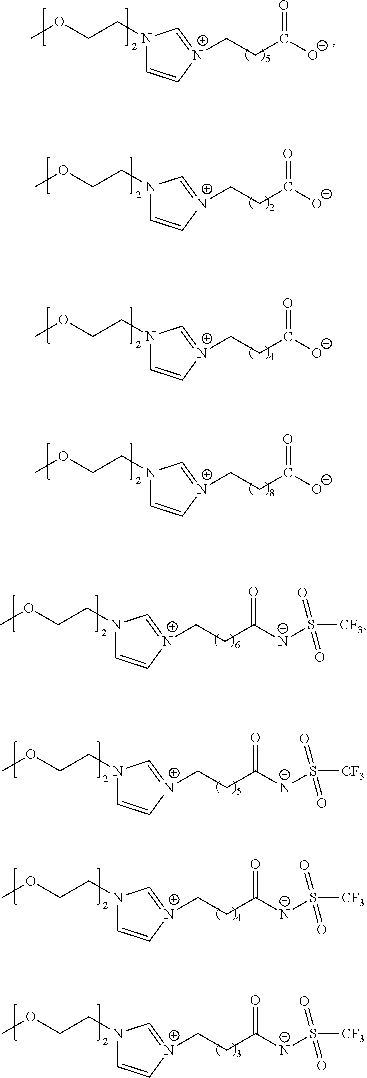

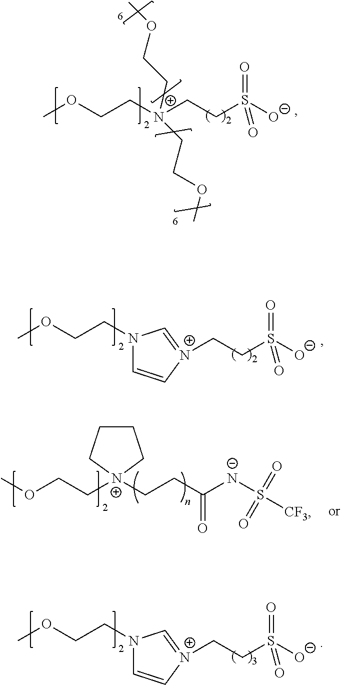

18. The method of claim 17, wherein the compound of Formula (I) is free of crystallization for about 1 to about 12 months at a temperature from about 20° C. to about 70° C.

19. The method of claim 10, wherein the compound of Formula (I) is formed with a yield from about 10% to about 100%.

20. The method of claim 19, wherein the yield is greater than about 40%.
